(19) United States
(12) Patent Application Publication
(10) Pub. No.: US 2003/0185796 A1
(43) Pub. Date: Oct. 2, 2003

(54) Methods of therapy for non-hodgkin's lymphoma

(75) Inventors: Maurice J. Wolin, Piedmont, CA (US); Sandra Milan, Orinda, CA (US); Deborah Hurst, Piedmont, CA (US)

Correspondence Address: Chiron Corporation, Intellectual Property - R440, P.O. Box 8097, Emeryville, CA, 94662-8097, US

(73) Assignee: Chiron Corporation, Emeryville, CA

(21) Appl. No.: 10/293,664

(22) Filed: Nov. 12, 2002

Related U.S. Application Data
(63) Continuation-in-part of application No. 09/815,597, filed on Mar. 23, 2001.
(60) Provisional application No. 60/192,047, filed on Mar. 24, 2000.

(51) Int. Cl. A61K 38/20; A61K 39/395

(52) U.S. Cl. 424/85.2; 424/144.1

(57) ABSTRACT

Methods for treating a human with lymphoma using a combination of interleukin-2 and at least one anti-CD20 antibody are provided. These therapeutic agents are administered as two separate pharmaceutical compositions, one containing IL-2, the other containing at least one anti-CD20 antibody, according to a dosing regimen. Administering of these two therapeutic agents together potentiates the effectiveness of either agent alone, resulting in a positive therapeutic response that is improved with respect to that observed with either agent alone. The therapeutic effects of these agents can be achieved using lower dosages of IL-2, thereby lessening the toxicity of prolonged IL-2 administration and the potential for tumor escape.

### CROSS-REFERENCE TO RELATED APPLICATION

This application is a continuation-in-part of U.S. application Ser. No. 09/815,597, filed Mar. 23, 2001, which claims the benefit of U.S. Provisional application Serial No. 60/192,047, filed Mar. 24, 2000, the contents of which are herein incorporated by reference in their entirety.

### FIELD OF THE INVENTION

The present invention is directed to methods of therapy for non-Hodgkin's lymphoma, more particularly to concurrent therapy with interleukin-2 and monoclonal antibodies targeting the CD20 B-cell surface antigen.

### BACKGROUND OF THE INVENTION

The non-Hodgkin's lymphomas are a diverse group of malignancies that are predominately of B-cell origin. In the Working Formulation classification scheme, these lymphomas been divided into low-, intermediate-, and high-grade categories by virtue of their natural histories (see “The Non-Hodgkin's Lymphoma Pathologic Classification Project,” Cancer 49(1982):2112-2135). The low-grade lymphomas are indolent, with a median survival of 5 to 10 years (Horning and Rosenberg (1984) N. Engl. J. Med. 311:1471-1475). Although chemotherapy can induce remissions in the majority of indolent lymphomas, cures are rare and most patients eventually relapse, requiring further therapy. The intermediate- and high-grade lymphomas are more aggressive tumors, but they have a greater chance for cure with chemotherapy. However, a significant proportion of these patients will relapse and require further treatment.

Interleukin-2 (IL-2) is a potent stimulator of natural killer (NK) and T-cell proliferation and function (Morgan et al. (1976) Science 193:1007-1011). This naturally occurring lymphokine has been shown to have anti-tumor activity against a variety of malignancies either alone or when combined with lymphokine-activated killer (LAK) cells or tumor-infiltrating lymphocytes (TIL) (see, for example, Rosenberg et al. (1987) N. Engl. J. Med. 316:889-897; Rosenberg (1988) Ann. Surg. 208:121-135; Topalian et al. 1988) J. Clin. Oncol. 6:839-853; Rosenberg et al. (1988) N. Engl. J. Med. 319:1676-1680; and Weber et al. (1992) J. Clin. Oncol. 10:33-40). The anti-tumor activity of IL-2 has best been described in patients with metastatic melanoma and renal cell carcinoma using Proleukin® IL-2, a commercially available IL-2 formulation. Other diseases, including lymphoma, also appear to respond to treatment with IL-2 (Gisselbrecht et al. (1994) Blood 83(8):2020-2022). However, high doses of IL-2 used to achieve positive therapeutic results with respect to tumor growth frequently cause severe side effects, including capillary leak, hypotension, and neurological changes (see, for example, Duggan et al. (1992) J. Immunotherapy 12:115-122; Gisselbrecht et al. (1994) Blood 83:2081-2085; and Sznol and Parkinson 1994) Blood 83:2020-2022).

Cancer research turned to the use of monoclonal antibodies as therapeutic agents. Produced in a similar fashion to diagnostic antibodies, therapeutic antibodies are designed to target tumor cells in order to facilitate their destruction. The use of therapeutic monoclonal antibodies has been hampered in the past primarily because of issues related to the antigenicity of the protein. Monoclonal antibodies have traditionally been a mouse product, and therefore generate an anti-murine response when injected into humans. This so-called HAMA (human anti-mouse antibody) response has imposed a great limitation on the use of monoclonal antibodies, as repeated dosing is nearly always precluded. In addition, serious complications, such as serum sickness, have been reported with the use of these agents. With the advent of chimeric and humanized antibodies, the therapeutic benefit of monoclonals is being realized. Using recombinant DNA technology, it is possible for a monoclonal antibody to be constructed by joining the variable or antigen recognition site of the antibody to a human backbone. This construction greatly decreases the incidence of blocking or clearing of the foreign antibodies from the host. This development allows for multiple doses of antibody to be given, providing the opportunity for reproducible and sustained responses with this therapy.

Monoclonal antibodies have increasingly become a method of choice for the treatment of lymphomas of the B-cell type. All B-cells express common cell surface markers, including CD20 and CD19. CD20 is a 33-37 kD phosphoprotein that is expressed early in B-cell differentiation and normally disappears in mature plasma cells. CD19 is closely associated with the B-cell antigen receptor and functions to send a signal when the cell engages antigen. CD20 and CD19 are expressed at very high levels on lymphoma cells. Approximately 90% of low-grade lymphomas express CD20 while CD19 is nearly ubiquitously expressed from all B-cells excluding bone marrow progenitors and plasma cells.

CD20 has become the premiere target for monoclonal therapy directed at B-cell antigens. In vitro work has demonstrated that monoclonal antibodies directed to CD20 result in cell death by apoptosis (Shan et al. (1998) Blood 91:1644-1652). Other studies report that B-cell death is primarily mediated by antibody-dependent cytotoxicity (ADCC). ADCC is a cellular mechanism that depends on specific effector cells carrying receptors for the monoclonal antibody bound to its target. These are in general receptors that are present on NK cells, neutrophils, and cells with monocyte/macrophage lineage. The NK cells appear to be the relevant mediators of this phenomenon, and antibodies to CD20 mediate their cytotoxicity primarily through ADCC.

Because of the possible immunological basis of the anti-tumor activity of anti-CD20 antibodies, combinations with other cytokines that enhance NK cell function have been examined. Cytokines such as IL-12, IL-15, TNF-alpha, TNF-beta, gamma-IFN, and IL-2 have been tested for potentiation of ADCC, a distinct NK function. All appear to be active in enhancing ADCC, although each agent is associated with its own specific toxicities.

The most compelling animal model is a nude mouse implanted with Daudi cells. Daudi cells are cells from a cell line derived from a patient with Burkitt's lymphoma, a B-cell tumor that expresses CD20. In this model, IL-2 was tested in combination with unconjugated anti-CD20 antibody both as a prophylaxis and after tumors had been established (Hooijberg et al. (1995) Cancer Research 55:2627-2634). The Hooijberg study showed that IL-2, in combination with unconjugated anti-CD20 antibody, is able to eliminate tumors completely in some animals. The combination was highly effective at affecting complete regression of tumors. Other cytokine combinations and the use of cytokines alone were much less effective in eliminating tumors. Hooijberg et al. also examined the combination in preventing tumor outgrowth and found that IL-2 and anti-CD20 were highly effective in preventing tumor growth.

Thus, this animal model supports the notion that IL-2 in combination with anti-CD20 is a potent mediator of B-cell tumor regression in prevention of tumor outgrowth. In this study, the IL-2 was given weekly and in a subcutaneous dose of 200,000 units/mouse. The equivalent dose in humans is as high as 600 MIU, which is greater than high-dose bolus used in treatment of renal cell carcinoma or metastatic melanoma.

Rituximab (IDEC-C2B8; IDEC Pharmaceuticals Corp., San Diego, Calif.) is a chimeric anti-CD20 monoclonal antibody containing human IgG1 and kappa constant regions with murine variable regions isolated from a murine anti-CD20 monoclonal antibody, IDEC-2B8 (Reff et al. (1994) Blood 83:435-445). The anti-lymphoma effects of rituximab are in part due to complement mediated cytotoxicity (CMC) antibody-dependent cell mediated cytotoxicity (ADCC), inhibition of cell proliferation, and finally direct induction of apoptosis. In early studies, rituximab induced a rapid depletion of CD20⁺ normal B-cells and lymphoma cells (Reff et al. (1994) Blood 83:435-445). Phase I trials of single doses up to 500 mg/m² and of 4 weekly doses of 375 mg/m² demonstrated clinical responses with no dose-limiting toxicity in low-grade or follicular lymphoma patients (Maloney et al. (1994) Blood 84:2457-2466. In a phase II trial, 4 weekly infusions of 375 mg/m² induced responses in 17 of 34 evaluable low-grade or follicular lymphoma patients, with a median time to progression of 10.2 months (Maloney et al. (1997) Blood 90:2188-2195). Side effects were in general associated with the first rituximab infusion and were mild to moderate. In a large pivotal phase II study, in 166 patients with low-grade or follicular lymphoma, objective response was reported for 76 (50%) of 151 evaluable patients and side effects were identical to those previously described (McLaughlin et al. (1998) J. Clin. Oncol. 16:2825-2833). Previous experience with rituximab in patients with the large cell histology is very limited, with fewer than 12 patients having been included in the early phase I and phase II studies. Recent studies, however, show that rituximab has activity in diffuse large cell and mantle cell lymphoma patients and should be tested in combination with chemotherapy in such patients (Coiffier et al (1998) Blood 92:1927-1932).

However, the reality of all current antineoplastic therapies is that tumors become resistant to therapy by a variety of mechanisms including tumor escape, acquired drug resistance, and down-regulation of cell-surface target molecules, among others. In a recent study, it was shown that therapy of B-cell lymphoma with anti-CD20 antibodies can result in loss of the CD20 antigen expression (Davis et al (1999) Clin. Cancer Res. 5:611-615). After two courses of therapy with rituximab, the subject developed a transformed lymphoma that no longer expressed CD20.

Thus, although IL-2 therapy alone and rituximab therapy alone have provided a means for partial treatment of lymphoma, new therapies are needed that will provide prolonged treatment for this cancer.

### BRIEF DESCRIPTION OF THE DRAWINGS

FIGS. 1A-1F shows the time course for natural killer (NK) cell count (CD 16/CD56 cells) (1A), CD4 cell count (1B), and CD8 cell count (1C) in 11 patients undergoing concurrent therapy with weekly rituximab therapy (375 mg/m2) and thrice-weekly doses of Proleukin® IL-2 for treatment of non-Hodgkin's lymphoma. Rituximab was administered by infusion over up to 6 hours on day 1 (D1), day 8 (D8), day 15 (D15), and day 22 (D22). Proleukin® IL-2 was administered subcutaneously three times per week for 4 weeks beginning on day 8. The doses of Proleukin® IL-2 were 4.5 MIU (3 patients), 10.0 MIU (3 patients), 14.0 MIU (3 patients), and 18.0 MIU (data shown for 2 patients). The corresponding cell counts for the 9 patients that have both tumor evaluation and week-10 lymphocyte subset counts available are shown in 1D (NK cell count), 1E (CD4 cell count) and 1F (CD8 cell count). PD=progressive disease; SD=stable disease; CR/PR=complete response or partial response.

FIG. 2 shows median NK cell counts at baseline, at 4-weeks post-initiation, and at 10-weeks post-initiation of concurrent therapy with Proleukin® IL-2 and rituximab for responders (complete response or partial response) versus non-responders (stable disease or progressive disease) from the study described for FIG. 1. Statistical significance was calculated using the Wilcoxon Rank Sum Test.

FIG. 3 shows the time course for natural killer (NK) cell activity using in vitro assays for NK cell function, including NK-mediated cytolytic function (NK), and LAK- and ADCC-mediated function (LAK and ADCC, respectively) as determined for a complete responder (CR). The CR patient participated in the thrice-weekly 18.0 MIU Proleukin® IL-2/weekly rituximab dosing regimen described for FIG. 1. See Example 1 below for details regarding functional assays. The data demonstrate that NK activity is maintained in a CR patient.

### SUMMARY OF THE INVENTION

Methods for providing treatment to a human subject with lymphoma using a combination of interleukin-2 (IL-2) or a variant thereof (hereinafter collectively “IL-2”) and at least one anti-CD20 antibody or a fragment thereof (hereinafter collectively “anti-CD20 antibody”) are provided. These two therapeutic agents are administered as separate pharmaceutical compositions, one containing IL-2, the other containing at least one anti-CD20 antibody, each according to a particular dosing regimen. The pharmaceutical composition comprising the anti-CD20 antibody is administered according to a weekly dosing schedule. The pharmaceutical composition comprising IL-2 is administered according to a twice- or thrice-weekly constant IL-2 dosing regimen, or is administered according to a two-level IL-2 dosing regimen. This two-level IL-2 dosing regimen comprises a first time period of IL-2 dosing, wherein a higher total weekly dose of IL-2 is administered to the subject, followed by a second time period of IL-2 dosing, wherein a lower total weekly dose of IL-2 is administered to the subject. The total weekly dose of IL-2 during the second time period of IL-2 dosing is lower than the total weekly dose of IL-2 administered during the first time period of IL-2 dosing. The total weekly dose to be administered during the first time period and/or during the second time period of IL-2 dosing can be administered as a single dose. Alternatively, the total weekly dose administered during either or both of the first and second time periods of IL-2 dosing can be partitioned into a series of equivalent doses that are administered according to a two-, three-, four-, five-, six- or seven-times-a-week dosing schedule. In some embodiments, multiple maintenance cycles of therapy with anti-CD20 antibody in combination with the two-level IL-2 dosing are administered to a subject in need of treatment for non-Hodgkin's lymphoma, wherein each maintenance cycle comprises administering the anti-CD20 antibody in combination with the two-level IL-2 dosing regimen. The need for administering these multiple maintenance cycles is assessed by monitoring natural-killer (NK) cell counts in subjects undergoing treatment with the methods of the invention. The methods also provide for an interruption in the two-level dosing regimen of IL-2, where the subject is given a time period off of IL-2 administration, or a time period off of IL-2 and anti-CD20 antibody administration, between the first and second time periods of the two-level IL-2 dosing regimen.

Administering of these two agents together in the manner set forth herein provides for greater therapeutic effectiveness than can be achieved using either of these agents alone, resulting in a positive therapeutic response that is improved with respect to that observed with either agent alone. In addition, the beneficial therapeutic effects of these agents can be achieved using lower cumulative dosages of IL-2, thereby lessening the toxicity of prolonged IL-2 administration and the potential for tumor escape.

A method for predicting clinical response of a subject undergoing a time period of concurrent therapy with anti-CD20 antibody and IL-2 is also provided. The method comprises monitoring natural killer (NK) cell expansion in said subject at about 1 week to about 14 weeks post-initiation of the time period of concurrent therapy. Threshold counts for NK cell expansion that are predictive of positive therapeutic response in a subject undergoing concurrent therapy with these two therapeutic agents are provided.

### DETAILED DESCRIPTION OF THE INVENTION

The present invention relates to methods of treating a human subject with lymphoma, more particularly non-Hodgkin's B-cell lymphoma. The methods comprise combination therapy with interleukin-2 or a variant thereof (hereinafter collectively “IL-2”) and at least one anti-CD20 antibody or a fragment thereof (hereinafter collectively “anti-CD20 antibody”). Combination therapy with these two therapeutic agents provides for anti-tumor activity. By “anti-tumor activity” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Subjects undergoing therapy with a combination of IL-2 and at least one anti-CD20 antibody in accordance with the methods of the present invention experience a physiological response that is beneficial with respect to treatment of B-cell lymphoma, more particularly non-Hodgkin's B-cell lymphoma.

The therapeutic methods of the invention are directed to treatment of any non-Hodgkin's B-cell lymphoma whose abnormal B-cell type expresses the CD20 surface antigen. By “CD20 surface antigen” is intended a 33-37 kD integral membrane phosphoprotein that is expressed during early pre-B cell development and persists through mature B-cells but which is lost at the plasma cell stage. Although CD20 is expressed on normal B cells, this surface antigen is usually expressed at very high levels on neoplastic B cells. More than 90% of B-cell lymphomas and chronic lymphocytic leukemias, and about 50% of pre-B-cell acute lymphoblastic leukemias express this surface antigen.

It is recognized that concurrent therapy with IL-2 and an anti-CD20 antibody may be useful in the treatment of any type of cancer whose unabated proliferating cells express the CD20 surface antigen. Thus, for example, where a cancer is associated with aberrant T-cell proliferation, and the aberrant T-cell population expresses the CD20 surface antigen, concurrent therapy in accordance with the methods of the invention would provide a positive therapeutic response with respect to treatment of that cancer. A human T-cell population expressing the CD20 surface antigen, though in reduced amounts relative to B-cells, has been identified (see Hultin et al. (1993) Cytometry 14:196-204).

It also is recognized that the methods of the invention are useful in the therapeutic treatment of B-cell lymphomas that are classified according to the Revised European and American Lymphoma Classification (REAL) system. Such B-cell lymphomas include, but are not limited to, lymphomas classified as precursor B-cell neoplasms, such as B-lymphoblastic leukemia/lymphoma; peripheral B-cell neoplasms, including B-cell chronic lymphocytic leukemia/small lymphocytic lymphoma, lymphoplasmacytoid lymphoma/immunocytoma, mantle cell lymphoma (MCL), follicle center lymphoma (follicular) (including diffuse small cell, diffuse mixed small and large cell, and diffuse large cell lymphomas), marginal zone B-cell lymphoma (including extranodal, nodal, and splenic types), hairy cell leukemia, plasmacytoma/myeloma, diffuse large cell B-cell lymphoma of the subtype primary mediastinal (thymic), Burkitt's lymphoma, and Burkitt's like high grade B-cell lymphoma; and unclassifiable low-grade or high-grade B-cell lymphomas.

By “non-Hodgkin's B-cell lymphoma” is intended any of the non-Hodgkin's based lymphomas related to abnormal, uncontrollable B-cell proliferation. For purposes of the present invention, such lymphomas are referred to according to the Working Formulation classification scheme (see “The Non-Hodgkin's Lymphoma Pathologic Classification Project,” Cancer 49(1982):2112-2135), that is those B-cell lymphomas categorized as low grade, intermediate grade, and high grade. Low-grade B-cell lymphomas include small lymphocytic, follicular small-cleaved cell, and follicular mixed small-cleaved cell lymphomas; intermediate-grade lymphomas include follicular large cell, diffuse small cleaved cell, diffuse mixed small and large cell, and diffuse large cell lymphomas; and high-grade lymphomas include large cell immunoblastic, lymphoblastic, and small non-cleaved cell lymphomas of the Burkitt's and non-Burkitt's type.

While the methods of the invention are directed to treatment of an existing non-Hodgkin's B-cell lymphoma, it is recognized that the methods may be useful in preventing further tumor outgrowths arising during therapy. The methods of the invention are particularly useful in the treatment of subjects having low-grade B-cell lymphomas, particularly those subjects having relapses following standard chemotherapy. Low-grade B-cell lymphomas are more indolent than the intermediate- and high-grade B-cell lymphomas and are characterized by a relapsing/remitting course. Thus, treatment of these lymphomas is improved using the methods of the invention, as relapse episodes are reduced in number and severity.

In accordance with the methods of the present invention, IL-2 and at least one anti-CD20 antibody as defined elsewhere below are used in combination to promote a positive therapeutic response with respect to non-Hodgkin's B-cell lymphoma. By “positive therapeutic response” is intended an improvement in the disease in association with the combined therapeutic activity of these agents, and/or an improvement in the symptoms associated with the disease. Thus, for example, an improvement in the disease may be characterized as a complete response. By “complete response” is intended an absence of clinically detectable disease with normalization of any previously abnormal radiographic studies, bone marrow, and cerebrospinal fluid (CSF). Such a response must persist for at least one month following treatment according to the methods of the invention. A complete response can be unconfirmed if no repeat evaluation of tumor status is done at least one month after the initial response is evaluated. Alternatively, an improvement in the disease may be categorized as being a partial response. By “partial response” is intended at least a 50% decrease in all measurable tumor burden (i.e., the number of tumor cells present in the subject) in the absence of new lesions and persisting for at least one month. Such a response is applicable to measurable tumors only. In addition to these positive therapeutic responses, the subject undergoing concurrent therapy with these two therapeutic agents may experience the beneficial effect of an improvement in the symptoms associated with the disease. Thus the subject may experience a decrease in the so-called B symptoms, i.e., night sweats, fever, weight loss, and/or urticaria.

Promotion of a positive therapeutic response with respect to a non-Hodgkin's lymphoma in a human subject is achieved via concurrent therapy with both IL-2 and at least one anti-CD20 antibody. By “concurrent therapy” is intended presentation of IL-2 and at least one anti-CD20 antibody to a human subject such that the therapeutic effect of the combination of both substances is caused in the subject undergoing therapy. Concurrent therapy may be achieved by administering at least one therapeutically effective dose of a pharmaceutical composition comprising IL-2 and at least one therapeutically effective dose of a pharmaceutical composition comprising at least one anti-CD20 antibody according to a particular dosing regimen. For example, in accordance with the methods of the present invention, concurrent therapy is achieved by administering the recommended total weekly doses of a pharmaceutical composition comprising IL-2 in combination with the recommended therapeutically effective doses of a pharmaceutical composition comprising at least one anti-CD20 antibody, each being administered according to a particular dosing regimen. By “therapeutically effective dose or amount” is intended an amount of the therapeutic agent that, when administered with a therapeutically effective dose or amount of the other therapeutic agent, brings about a positive therapeutic response with respect to treatment of a B-cell lymphoma such as non-Hodgkin's lymphoma. Administration of the separate pharmaceutical compositions can be at the same time (i.e., simultaneously) or at different times (i.e., sequentially, in either order, on the same day, or on different days), so long as the therapeutic effect of the combination of both substances is caused in the subject undergoing therapy.

The separate pharmaceutical compositions comprising these therapeutic agents as therapeutically active components may be administered using any acceptable method known in the art. Thus, for example, the pharmaceutical composition comprising IL-2 can be administered by any form of injection, including intravenous (IV), intramuscular (IM), or subcutaneous (SC) injection. In some embodiments of the invention, the pharmaceutical composition comprising IL-2 is administered by SC injection. In other embodiments of the invention, the pharmaceutical composition comprising IL-2 is a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release IL-2 pharmaceutical composition. The pharmaceutical composition comprising the anti-CD20 antibody is administered, for example, intravenously. When administered intravenously, the pharmaceutical composition comprising the anti-CD20 antibody can be administered by infusion over a period of about 1 to about 10 hours. In some embodiments, infusion of the antibody occurs over a period of about 2 to about 8 hours, over a period of about 3 to about 7 hours, over a period of about 4 to about 6 hours, or over a period of about 6 hours, depending upon the anti-CD20 antibody being administered.

Concurrent therapy with both of these therapeutic agents in the manner set forth herein provides for greater therapeutic effectiveness than can be achieved using either of these agents alone, resulting in a positive therapeutic response that is improved with respect to that observed with either agent alone. This positive therapeutic response is achieved using lower cumulative doses of IL-2 than would be required to get similar therapeutic benefit using IL-2 as a single agent. Thus, a dose of IL-2 alone that is not normally therapeutically effective may be therapeutically effective when administered in combination with at least one anti-CD20 antibody in accordance with the methods of the invention. The significance of this is two-fold. First, the potential therapeutic benefits of treatment of lymphoma with IL-2 can be realized at IL-2 doses that minimize toxicity responses normally associated with prolonged IL-2 therapy or high-bolus IL-2 administration. Such toxicity responses include, but are not limited to, chronic fatigue, nausea, hypotension, fever, chills, weight gain, pruritis or rash, dysprea, azotemia, confusion, thrombocytopenia, myocardial infarction, gastrointestinal toxicity, and vascular leak syndrome (see, for example, Allison et al. (1989) J. Clin. Oncol. 7(l):75-80). Secondly, targeting of specific molecules on a tumor cell surface by monoclonal antibodies can select for clones that are not recognized by the antibody or are not affected by its binding, resulting in tumor escape, and loss of effective therapeutic treatment. Such tumor escape has been documented with repeated doses of an anti-CD20 antibody (Davis et al. (1999) Clin. Cancer Res. 5:611-615). The improved therapeutic benefit of anti-CD20 antibodies administered in combination with IL-2 may translate into less frequent administration of monoclonal antibodies, thereby lessening the potential for tumor escape.

The amount of at least one anti-CD20 antibody to be administered in combination with an amount of IL-2, and the amount of either of these therapeutic agents needed to potentiate the effectiveness of the other therapeutic agent, are readily determined by one of ordinary skill in the art without undue experimentation given the disclosure set forth herein. Factors influencing the respective amount of IL-2 to be administered in combination with a given amount of at least one anti-CD20 antibody in accordance with the dosing regimens disclosed herein include, but are not limited to, the mode of administration, the particular lymphoma undergoing therapy, the severity of the disease, the history of the disease, and the age, height, weight, health, and physical condition of the individual undergoing therapy. Similarly, these factors will influence the necessity for repeated exposure to combination IL-2/anti-CD20 antibody therapy in the manner set forth herein. Generally, a higher dosage of the antibody agent is preferred with increasing weight of the subject undergoing therapy.

In accordance with the methods of the present invention, the human subject undergoing treatment with one or more weekly doses of anti-CD20 antibody as defined herein below is also administered IL-2 as defined herein below according to a constant IL-2 dosing regimen or according to a two-level IL-2 dosing regimen. The first therapeutically effective dose administered to the subject can be the anti-CD20 antibody or can be the IL-2, depending upon which IL-2 dosing regimen is used. Generally, where the individual is to receive a constant IL-2 dosing regimen, the initial therapeutic agent to be administered is anti-CD20 antibody, while the first dose of IL-2 is administered subsequently, for example, within 10 days following administration of the first therapeutically effective dose of the anti-CD20 antibody, for example, within 1, 2, 3, 4, 5, 6, 7, 8, 9, or 10 days. In some embodiments, the constant IL-2 dosing regimen is initiated by administering a first dose of IL-2 within 7 days of administering the first therapeutically effective dose of anti-CD20 antibody, such as within 1, 2, 3, 4, 5, 6, or 7 days. Where the individual is to receive a two-level IL-2 dosing regimen, either therapeutic agent can be administered first, so long as the subject has an overlapping period of time during which both therapeutic agents are being administered to the subject, each according to the particular dosing regimen disclosed herein.

Thus, in one embodiment, the two-level IL-2 dosing regimen is initiated prior to initiating weekly administration of therapeutically effective doses of anti-CD20 antibody. In this manner, a first dose of IL-2 is administered up to one month before the first dose of anti-CD20 antibody is administered. By “up to one month” is intended the first dose of IL-2 is administered at least one day before initiating anti-CD20 antibody administration, but not more than one month (i.e., 30 days) before initiating anti-CD20 antibody administration. Thus, IL-2 administration can begin, for example, 1 day, 2 days, 3 days, 4 days, 5 days, 6 days, 7 days (i.e., 1 week), 10 days, 14 days (i.e., two weeks), 17 days, 21 days (i.e., 3 weeks), 24 days, 28 days (4 weeks), or up to one month (i.e., 30 days) before administering the first therapeutically effective dose of the anti-CD20 antibody.

In other embodiments, the two-level IL-2 dosing regimen and anti-CD20 antibody administration begin concurrently on the same day, either at the same time (i.e., simultaneous administration) or at different times (i.e., sequential administration, in either order). Thus, for example, in one embodiment where concurrent therapy with these two therapeutic agents begins on day 1 of a treatment period, a first therapeutically effective dose of anti-CD20 antibody and a first dose of IL-2 would both be administered on day 1 of this treatment period.

In alternative embodiments, a first therapeutically effective dose of anti-CD20 antibody is administered to the subject, for example, on day 1 of a treatment period, and the two-level IL-2 dosing regimen is initiated by administering a first dose of IL-2 within 10 days of administering the first therapeutically effective dose of anti-CD20 antibody. In such embodiments, preferably the two-level IL-2 dosing regimen is initiated by administering a first dose of IL-2 within 7 days of administering the first therapeutically effective dose of anti-CD20 antibody, such as within 1, 2, 3, 4, 5, 6, or 7 days. Depending upon the severity of the disease, the patient's health, and prior history of the patient's disease, repeated sessions of concurrent therapy with IL-2 and anti-CD20 antibody in accordance with the dosing regimens disclosed herein is contemplated. Such repeated sessions are referred to herein as maintenance cycles, which are described in more detail below.

In accordance with the methods of the present invention, a therapeutically effective dose of anti-CD20 antibody is administered weekly in combination with a constant IL-2 dosing regimen or in combination with a two-level IL-2 dosing regimen. The duration of weekly administration of a therapeutically effective dose of anti-CD20 antibody and the duration of either of the IL-2 dosing regimens will depend upon the subject's overall health, history of disease progression, and tolerance of the particular anti-CD20/IL-2 administration protocol. Generally, the duration of weekly anti-CD20 antibody administration is about 4 weeks to about 8 weeks, including 4, 5, 6, 7, or 8 weeks. The duration of IL-2 administration is a function of the IL-2 dosing regimen used.

In some embodiments of the invention, the subject undergoing concurrent therapy with these two therapeutic agents is administered a constant IL-2 dosing regimen. By “constant IL-2 dosing regimen” is intended the subject undergoing concurrent therapy with IL-2 and anti-CD20 antibody is administered a constant total weekly dose of IL-2. This total weekly dose of IL-2 is partitioned into a series of equivalent doses that are administered according to a two- or three-times-a-week dosing schedule. With respect to the constant IL-2 dosing regimen, a “two-times-a-week,” “twice weekly,” or “two times per week” dosing schedule is intended to mean that the total weekly dose of IL-2 is partitioned into two equivalent doses that are administered to the subject within a 7-day period, allowing for a minimum of 72 hours between doses and a maximum of 96 hours between doses. By “three-times-a-week,” “thrice weekly,” or “three times per week” dosing schedule is intended the constant total weekly dose of IL-2 is partitioned into three equivalent doses that are administered to the subject within a 7-day period, allowing for a minimum of 25 hours between doses and a maximum of 72 hours between doses. The duration of the constant IL-2 dosing regimen is about 4 weeks to about 10 weeks, for example, 4, 5, 6, 7, 8, 9, or 10 weeks.

Thus, in one such embodiment, concurrent therapy with these two therapeutic agents comprises weekly administration of a therapeutically effective dose of at least one anti-CD20 antibody for a period of 4 weeks in combination with administration of a 4-week to 8-week constant IL-2 dosing regimen, wherein each recommended total weekly dose of IL-2 is partitioned into three equivalent doses that are administered to the subject within a 7-day period according to a three-times-a-week dosing schedule (i.e., allowing for a minimum of 25 hours between doses and a maximum of 72 hours between doses). In one embodiment, the constant IL-2 dosing regimen has a duration of 4 weeks; in another embodiment, the constant IL-2 dosing regimen has a duration of 8 weeks. For example, a therapeutically effective dose of at least one anti-CD20 antibody is administered on days 1, 8, 15, and 22 of a treatment period, and a 4-week or 8-week constant IL-2 dosing regimen is initiated on day 3, 4, 5, 6, 7, 8, 9, or 10 of the same treatment period. In one such embodiment, the 4-week or 8-week constant IL-2 dosing regimen begins on day 8 of the same treatment period, with each recommended total weekly dose of IL-2 being partitioned into three equivalent doses that are administered according to a three-times-a-week dosing schedule (i.e., 4 total weekly doses of IL-2, which are administered as a total of 12 equivalent doses; or 8 total weekly doses of IL-2, which are administered as a total of 24 equivalent doses).

Thus, for example, where a 4-week constant IL-2 dosing regimen is contemplated, therapeutically effective doses of anti-CD20 antibody are administered on days 1, 8, 15, and 22 of a treatment period, while the 12 equivalent doses of IL-2 are administered on days 8, 10, 12, 15, 17, 19, 22, 24, 26, 29, 31, and 33 of the same treatment period. As the three equivalent doses of IL-2 that are to be administered each week are staggered to allow for a minimum of 25 hours between IL-2 doses and a maximum of 72 hours between IL-2 doses, the three equivalent doses within any given week can be administered on, for example, days 1, 2, and 5 of any given week of IL-2 administration; on days 1, 3, and 5 of any given week; on days 1, 3, and 6 of any given week; on days 1, 4, and 5 of any given week; on days 1, 4, and 6 of any given week; or on days 1, 4, and 7 of any given week; so long as the time period between IL-2 doses is a minimum of 25 hours and a maximum of 72 hours, and the entire constant total weekly dose of IL-2 is administered.

In another embodiment of the invention, a similar dosing regimen is used, with the exception of administering the weekly therapeutically effective doses of at least one anti-CD20 antibody for a total of 8 weeks in combination with a constant IL-2 dosing regimen that has a duration of about 4 weeks to about 10 weeks, including 4, 5, 6, 7, 8, 9, or 10 weeks. In this embodiment, concurrent therapy with these two therapeutic agents comprises administration of a therapeutically effective dose of at least one anti-CD20 antibody on days 1, 8, 15, 22, 29, 36, 43, and 50 of a treatment period, for a total of 8 therapeutically effective doses of anti-CD20 antibody, and initiation of the 4-week to 10-week constant IL-2 dosing regimen beginning on day 3, 4, 5, 6, 7, 8, 9, or 10 of the same treatment period, where each recommended total weekly dose of IL-2 is partitioned into three equivalent doses that are administered according to a three-times-a-week dosing schedule as described above. In one embodiment, the constant IL-2 dosing regimen begins on day 8 (i.e., at the start of week 2) of the treatment period, and continues over 8 consecutive weeks (i.e., the recommended constant total weekly dose is administered for weeks 2-9) of the same treatment period.

In each of the foregoing embodiments, the recommended total weekly dose of IL-2 that is to be administered over the 4-week to 10-week constant IL-2 dosing regimen can alternately be partitioned into two equivalent doses that are administered according to a two-times-a-week dosing schedule. In this manner, during each week of the constant IL-2 dosing regimen, the two equivalent doses are administered each week, beginning on day 1 of the first week of IL-2 administration, with a minimum of 72 hours between doses and a maximum of 96 hours between doses. Thus, for example, if the 4-week to 10-week constant IL-2 dosing regimen begins on day 8 of a treatment period (i.e., 8 days after the first therapeutically effective dose of anti-CD20 antibody is administered), the second dose for that week can be administered on day 11 or day 12 of the treatment period, with the next therapeutically effective dose of IL-2 being administered on day 15 of this same treatment period.

Thus, in one embodiment of the invention, concurrent therapy with these two therapeutic agents comprises administration of a therapeutically effective dose of at least one anti-CD20 antibody on days 1, 8, 15, 22, 29, 36, 43, and 50 of a treatment period, for a total of 8 therapeutically effective doses of anti-CD20 antibody, with initiation of the 4-week to 10-week constant IL-2 dosing regimen beginning on day 3, 4, 5, 6, 7, 8, 9, or 10 of the same treatment period, where each of the recommended total weekly doses of IL-2 are partitioned into two equivalent doses that are administered according to a two-times-a-week dosing schedule. In one such embodiment, the constant IL-2 dosing regimen begins on day 8 (i.e., at the start of week 2) of the treatment period, and has a duration of 4 weeks (i.e., 4 total weekly doses of IL-2, which are administered as a total of 8 equivalent doses) or 8 weeks (i.e., 8 total weekly doses of IL-2, which are administered as a total of 16 equivalent doses). Thus, for example, where IL-2 administration occurs over an 8-week period and begins 1 week following administration of the first therapeutically effective dose of anti-CD20 antibody, the complete treatment period occurs over 9 weeks. In an alternative embodiment, this 4-week or 8-week constant IL-2 dosing regimen is followed (i.e., recommend total weekly dose of IL-2 administered during weeks 2-5 or weeks 2-9, respectively, of a treatment period), while a therapeutically effective dose of the anti-CD20 antibody is administered once a week over the first 4 weeks of the treatment period, i.e., on days 1, 8, 15, and 22, for a total of 4 therapeutically effective doses of the antibody anti-tumor agent.

In other embodiments of the invention, concurrent therapy with these two therapeutic agents comprises a “two-level IL-2 dosing regimen.” By “two-level IL-2 dosing regimen” is intended the subject undergoing concurrent therapy with IL-2 and anti-CD20 antibody is administered IL-2 during two time periods of IL-2 dosing, which have a combined duration of about 4 weeks to about 16 weeks, including, 4, 5, 6, 7, 8, 9, 10, 11, 12, 13, 14, 15, or 16 weeks. In one embodiment, the two-level IL-2 dosing regimen has a combined duration of about 4 weeks to about 12 weeks; in other embodiments, the two-level IL-2 dosing regimen has a combined duration of about 4 weeks to about 8 weeks, including about 4, 5, 6, 7, or 8 weeks. The total weekly dose of IL-2 that is to be administered during the first and second time periods of the two-level IL-2 dosing regimen is chosen such that a higher total weekly dose of IL-2 is given during the first time period and a lower total weekly dose of IL-2 is given during the second time period. The duration of the individual first and second time periods of the two-level IL-2 dosing regimen can vary, depending upon the health of the individual and history of disease progression. Generally, the subject is administered higher total weekly doses of IL-2 for at least 1 week out of the 4-week to 16-week two-level IL-2 dosing regimen. In one embodiment, higher total weekly doses of IL-2 are administered during the first half of the two-level IL-2 dosing regimen, with lower total weekly doses being administered during the second half of the two-level IL-2 dosing regimen. Thus, for example, where the two-level IL-2 dosing regimen has a combined duration of 8 weeks, the higher total weekly doses of IL-2 would be administered for the first 4 weeks of IL-2 dosing, and the lower total weekly doses of IL-2 would be administered for the second 4 weeks of IL-2 dosing.

Though specific dosing regimens are disclosed herein below, it is recognized that the invention encompasses any administration protocol that provides for concurrent therapy with an anti-CD20 antibody and a two-level IL-2 dosing regimen that provides for initial exposure to higher total weekly doses of IL-2, and subsequent exposure to lower total weekly doses of IL-2. While not being bound by theory, it is believed that administering a higher dose of IL-2 during the initial stages of IL-2 dosing provides for an initial stimulation of NK cell activity that can be maintained by a lower dose during the subsequent weeks of IL-2 dosing. As IL-2 side effects are dose-related, the lowered dose will increase tolerability during the extended treatment period.

Thus, the methods of the invention contemplate treatment regimens where a therapeutically effective dose of at least one anti-CD20 antibody is administered once a week for one or more weeks, for example, for 4 weeks or 8 weeks, in combination with a two-level IL-2 dosing regimen having a combined duration of about 4 weeks to about 16 weeks, including 4, 5, 6, 7, 8, 9, 10, 11, 12, 13, 14, 15, or 16 weeks. Either agent could be administered first, as explained above for this two-level IL-2 dosing regimen. For example, in one embodiment, a therapeutically effective dose of anti-CD20 antibody is administered first, for example, on day 1 of a treatment period, followed by initiation of the two-level IL-2 dosing regimen within 10 days, preferably within 7 days of the first administration of the anti-CD20 antibody, for example, within 1, 2, 3, 4, 5, 6, or 7 days. During the two-level IL-2 dosing regimen, a higher total weekly dose of IL-2 is administered in the first time period of the two-level IL-2 dosing regimen, for example, over the first 1-4 weeks of IL-2 administration, and lower total weekly doses of IL-2 are administered during the second time period of the two-level IL-2 dosing regimen (i.e., over the remaining course of the two-level IL-2 dosing regimen).

In one embodiment, the methods of the invention provide for weekly administration of a therapeutically effective dose of a pharmaceutical composition comprising at least one anti-CD20 antibody over a period of 4 weeks in combination with a two-level IL-2 dosing regimen having a combined duration of 4 weeks to 8 weeks, including 4, 5, 6, 7, or 8 weeks. In this manner, a therapeutically effective dose of at least one anti-CD20 antibody is administered on days 1, 8, 15, and 22 of a treatment period, and the 4-week to 8-week two-level IL-2 dosing regimen is initiated beginning on day 3, 4, 5, 6, 7, 8, 9, or 10 of the same treatment period.

In one such embodiment, therapeutically effective doses of the pharmaceutical composition comprising the anti-CD20 antibody are administered weekly for 4 weeks beginning on day 1 of a treatment period and the two-level IL-2 dosing regimen begins on day 8 of the same treatment period and continues for 8 weeks (i.e., during weeks 2-9 of the treatment period). In an alternative embodiment, this 8-week two-level IL-2 dosing regimen is followed (i.e., IL-2 administration occurring during weeks 2-9 of a treatment period), while a therapeutically effective dose of the pharmaceutical composition comprising the anti-CD20 antibody is administered once a week over the first 8 weeks of the treatment period (i.e., on day 1, 8, 15, 22, 29, 36, 43, and 50 of the treatment period).

For human subjects undergoing concurrent therapy with weekly administration of a therapeutically effective dose of anti-CD20 antibody in combination with a two-level IL-2 dosing regimen, the total weekly dose of IL-2 during the first and second time periods of this two-level IL-2 dosing regimen can be administered as a single dose, or can be partitioned into a series of equivalent doses that are administered according to a two- three-, four-, five-, six-, or seven-times-a-week dosing schedule. Thus, for example, the higher total weekly dose during the first time period can be administered as a single dose, or can be partitioned into a series of equivalent doses that are administered according to a two- three-, four-, five-, six-, or seven-times-a-week dosing schedule. Similarly, the lower total weekly dose during the second time period can be administered as a single dose, or can be partitioned into a series of equivalent doses that are administered according to a two-, three-, four-, five-, six-, or seven-times-a-week dosing schedule. For purposes of the two-level IL-2 dosing regimen, a “two-, three-, four-, five-, six-, or seven-times-a-week dosing schedule” is intended to mean that the total weekly dose is partitioned into two, three, four, five, six, or seven equivalent doses, respectively, which are administered to the subject over the course of a 7-day period, with no more than one equivalent dose being administered per 24-hour period. The series of equivalent doses can be administered on sequential days, or can be administered such that one or more days occur between any two consecutive doses, depending upon the total number of equivalent doses administered per week.

Thus, for example, where a series of two equivalent doses of IL-2 are administered per week (i.e., over a 7-day period) and the first equivalent dose of that week is administered on day 1, the second equivalent dose of IL-2 can be administered on day 2, 3, 4, 5, 6, or 7 of that week. In one embodiment, the total weekly dose of IL-2 is partitioned into two equivalent doses that are administered to the subject within a 7-day period, allowing for a minimum of 72 hours between doses and a maximum of 96 hours between doses.

Similarly, where a series of three equivalent doses of IL-2 are administered per week and the first equivalent dose of that week is administered on day 1, the second equivalent dose can be administered on day 2, 3, 4, 5, or 6 of that week, and the third equivalent dose can be administered on day 3, 4, 5, 6, or 7 of that week, so long as about 24 hours occur between administration of the second and third equivalent doses. In one embodiment, the total weekly dose of IL-2 is partitioned into three equivalent doses that are administered to the subject within a 7-day period, allowing for a minimum of 25 hours between doses and a maximum of 72 hours between doses.

Where a series of four equivalent doses of IL-2 are administered per week and the first equivalent dose of that week is administered on day 1, the second equivalent dose can be administered on day 2, 3, 4, or 5 of that week, the third equivalent dose can be administered on day 3, 4, 5, or 6 of that week, and the fourth equivalent dose can be administered on day 4, 5, 6, or 7 of that week, so long as about 24 hours occur between administration of any two consecutive doses (i.e., between the first and second equivalent doses, between the second and third equivalent doses, and between the third and fourth equivalent doses).

Where a series of five equivalent doses are administered per week and the first equivalent dose of that week is administered on day 1, the second equivalent dose can be administered on day 2, 3, or 4 of that week, the third equivalent dose can be administered on day 3, 4, or 5 of that week, the fourth equivalent dose can be administered on day 4, 5, or 6 of that week, and the fifth equivalent dose can be administered on day 5, 6, or 7 of that week, so long as about 24 hours occur between administration of any two consecutive doses (i.e., between the first and second equivalent doses, between the second and third equivalent doses, between the third and fourth equivalent doses, and between the fourth and fifth equivalent doses).

Where a series of six equivalent doses of IL-2 are administered per week and the first equivalent dose of that week is administered on day 1, the second equivalent dose can be administered on day 2 or 3 of that week, the third equivalent dose can be administered on day 3 or 4 of that week, the fourth equivalent dose can be administered on day 4 or 5 of that week, the fifth equivalent dose can be administered on day 5 or 6 of that week, and the sixth equivalent dose can be administered on day 6 or 7 of that week, so long as about 24 hours occur between administration of any two consecutive doses (i.e., between the first and second equivalent doses, between the second and third equivalent doses, between the third and fourth equivalent doses, between the fourth and fifth equivalent doses, and between the fifth and sixth equivalent doses).

In one embodiment, the total weekly dose of IL-2 is partitioned into seven equivalent doses, which are administered daily over the 7-day period, with about 24 hours occurring between each consecutive dose.

It is not necessary that the same dosing schedule be followed for the first and second periods of the two-level IL-2 dosing regimen. Thus, the dosing schedule can be adjusted to accommodate an individual's tolerance of prolonged IL-2 therapy in combination with anti-CD20 antibody therapy, and to reflect the individual's responsiveness to concurrent therapy with these two therapeutic agents. The preferred dosing schedule during these two time periods is readily determined by the managing physician given the patient's medical history and the guidance provided herein.

Thus, the present invention provides methods for treating a human subject with non-Hodgkin's lymphoma using concurrent therapy with weekly administration of a therapeutically effective dose of anti-CD20 antibody in combination with either a constant IL-2 dosing regimen or a two-level IL-2 dosing regimen. For purposes of the present invention, the therapeutically effective dose of at least one anti-CD20 antibody to be administered weekly is in the range from about 100 mg/m² to about 550 mg/m², about 125 mg/m² to about 500 mg/m², about 225 mg/m² to about 400 mg/m², or about 375 mg/m². The pharmaceutical composition comprising the anti-CD20 antibody is administered, for example, intravenously, as noted herein above. The IL-2 is administered, for example, by IV, IM, or SC injection, in combination with the anti-CD20 antibody therapy so as to provide the recommended total weekly doses of IL-2 during the constant IL-2 dosing regimen or during the two-level IL-2 dosing regimen as described more fully below. The following embodiments provide guidance as to suitable dosing regimens, though any number of different dosing regimens can be contemplated by one of skill in the art apprised of the disclosure set forth herein.

For purposes of the following discussion of total weekly doses of IL-2 to be administered during the constant or two-level IL-2 dosing regimen, the multimeric IL-2 pharmaceutical composition commercially available under the tradename Proleukin® IL-2 (Chiron Corporation, Emeryville, Calif.) is used as the reference IL-2 standard. By “reference IL-2 standard” is intended the formulation of IL-2 that serves as the basis for determination of the total weekly IL-2 doses to be administered to a human subject with lymphoma in accordance with the desired constant or two-level IL-2 dosing regimen in combination with at least one anti-CD20 antibody to achieve the desired positive effect, i.e., a positive therapeutic response that is improved with respect to that observed with either of these therapeutic agents alone.

Where Proleukin® IL-2 is to be administered according to a constant IL-2 dosing regimen, the total weekly dose is about 30.0 MIU to about 54.0 MIU, depending upon the duration of the treatment period and whether the IL-2 is dosed on a twice-a-week or thrice-a-week dosing schedule, while the therapeutically effective dose of anti-CD20 antibody to be administered weekly is in the range from about 100 mg/m² to about 550 mg/m², about 125 mg/m² to about 500 mg/m², about 225 mg/m² to about 400 mg/m², or about 375 mg/m². Thus, for example, in some embodiments, the total amount of Proleukin® IL-2 that is to be administered per week as part of a constant IL-2 dosing regimen is about 30.0 MIU, 32.0 MIU, 35.0 MIU, 37.0 MIU, 40.0 MIU, 42.0 MIU, 45.0 MIU, 47.0 MIU, 50.0 MIU, 52.0 MIU, or 54.0 MIU, and the total amount of anti-CD20 antibody is about 225, 250, 275, 300, 325, 350, 375, or 400 mg/m²/weekly dose. When the total weekly dose of Proleukin® IL-2 is about 30.0 MIU to about 42.0 MIU, the total amount of anti-CD20 antibody is about 325, 350, 375, or 400 mg/m²/weekly dose. In one embodiment, the total weekly dose of Proleukin® IL-2 is about 42.0 MIU, and the total amount of anti-CD20 antibody is about 375 mg/m²/weekly dose. As previously noted, the total weekly dose of IL-2 during a constant IL-2 dosing regimen is partitioned into two or three equivalent doses that are administered according to a two- or three-times-a-week dosing schedule, respectively. Thus, for example, where the total weekly dose of Proleukin® IL-2 is 30.0 MIU, the three equivalent doses of this reference IL-2 standard to be administered during each week would be 10.0 MIU, and the two equivalent doses of this reference IL-2 standard to be administered during each week would be 15.0 MIU. Similarly, where the total weekly dose of Proleukin® IL-2 is 54.0 MIU, the three equivalent doses of this reference IL-2 standard to be administered during each week would be 18.0 MIU, and the two equivalent doses of this reference IL-2 standard to be administered during each week would be 27.0 MIU.

Where Proleukin® IL-2 is to be administered according to a two-level IL-2 dosing regimen, the higher total weekly dose that is administered during the first time period of this dosing regimen is about 30.0 MIU to about 54.0 MIU, and the lower total weekly dose that is administered during the second time period of this dosing regimen is about 18.0 MIU to about 39.0 MIU. As previously noted, the total weekly dose administered during the first time period of the two-level IL-2 dosing regimen, for example, during the first half of this dosing regimen, is always higher than the total weekly dose administered during the second time period of the two-level IL-2 dosing regimen, for example, during the second half of this dosing regimen.

Thus, in some embodiments, the higher total weekly dose of Proleukin®IL-2 that is administered during the first time period of the two-level IL-2 dosing regimen is about 30.0 MIU to about 54.0 MIU, including about 30.0 MIU, 32.0 MIU, 35.0 MIU, 37.0 MIU, 40.0 MIU, 42.0 MIU, 45.0 MIU, 47.0 MIU, 50.0 MIU, 52.0 MIU, or 54.0 MIU, and other such values falling within this higher dosing range; and the lower total weekly dose of Proleukin®IL-2 is about 18.0 MIU to about 39.0 MIU, including 18.0 MIU, 20.0 MIU, 23.0 MIU, 25.0 MIU, 27.0 MIU, 30.0 MIU, 32 MIU, 35.0 MIU, 37.0 MIU, and 39.0 MIU, and other such values falling within this lower dosing range. In one embodiment, the two-level IL-2 dosing regimen has a combined duration of 4 weeks to 8 weeks, where the higher total weekly dose of Proleukin®IL-2 that is administered during the first time period of the two-level IL-2 dosing regimen is about 30.0 MIU to about 42.0 MIU, such as 30.0 MIU, 32.0 MIU, 34.0 MIU, 36.0 MIU, 38.0 MIU. 40.0 MIU, and 42.0 MIU, and the lower total weekly dose of Proleukin®IL-2 that is administered during the second time period of the two-level IL-2 dosing regimen is about 18.0 MIU to about 30.0 MIU, such as 18.0, 20.0, 22.0, 24.0, 26.0, 28.0, and 30.0 MIU. In one such embodiment, the higher total weekly dose of Proleukin®IL-2 that is administered during the first time period is 42.0 MIU and the lower total weekly dose of Proleukin®IL-2 that is administered during the second time period is 30.0 MIU. As previously noted, the total weekly dose of IL-2 during the first and second time periods of a two-level IL-2 dosing regimen is administered as a single dose, or is partitioned into a series of equivalent doses that are administered according to a two-, three-, four-, five-, six-, or seven-times-a-week dosing schedule. Thus, for example, where the total weekly dose of Proleukin® IL-2 during the first period of the two-level IL-2 dosing regimen is 42.0 MIU, the three equivalent doses of this reference IL-2 standard to be administered during each week would be 14.0 MIU, and the two equivalent doses of this reference IL-2 standard to be administered during each week would be 21.0 MIU. Similarly, where the total weekly dose of Proleukin® IL-2 during the second period of the two-level IL-2 dosing regimen is is 30.0 MIU, the three equivalent doses of this reference IL-2 standard to be administered during each week would be 10.0 MIU, and the two equivalent doses of this reference IL-2 standard to be administered during each week would be 15.0 MIU.

In accordance with the methods of the present invention, the subject is administered this two-level IL-2 dosing regimen in combination with weekly administration of a therapeutically effective dose of anti-CD20 antibody. The therapeutically effective dose of anti-CD20 antibody to be administered weekly is in the range from about 100 mg/m² to about 550 mg/m², about 125 mg/m² to about 500 mg/m², about 225 mg/m² to about 400 mg/m², or about 375 mg/m². Thus, for example, in some embodiments, the total amount of anti-CD20 antibody is about 225, 250, 275, 300, 325, 350, 375, or 400 mg/m²/weekly dose. In other embodiments, the total amount of anti-CD20 antibody is about 325, 350, 375, or 400 mg/m²/weekly dose.

In a preferred embodiment, the therapeutically effective dose of anti-CD20 antibody is administered once a week for 4 weeks or 8 weeks beginning on day 1 of a treatment period, and the two-level IL-2 dosing regimen is initiated on day 8 of this treatment period and has a combined duration of 8 weeks. In this embodiment, the higher total weekly dose of IL-2 administered during weeks 2-5 of the treatment period is about 30.0 MIU to about 54.0 MIU, preferably about 30.0 MIU to about 42.0 MIU, and the lower total weekly dose of IL-2 administered during weeks 6-9 is about 18.0 MIU to about 39.0 MIU, preferably about 18.0 MIU to about 30.0 MIU. The higher and lower total weekly doses of IL-2 are administered as a single dose, or are partitioned into equivalent doses that are administered according to a two-, three-, four-, five-, six-, or seven-times-a-week dosing schedule. In one such embodiment, the higher total weekly dose of IL-2 during weeks 2-5 of the treatment period is about 30.0 MIU to about 42.0 MIU, for example, 42.0 MIU, and the lower total weekly dose or IL-2 is about 18.0 MIU to about 30.0 MIU, for example, 30.0 MIU. In this embodiment, each of the higher and lower total weekly doses of IL-2 are partitioned into two equivalent doses that are administered according to a two-times-a-week dosing schedule, where the two equivalent doses are administered to the subject within a 7-day period, allowing for a minimum of 72 hours between doses and a maximum of 96 hours between doses. In an alternative embodiment, each of the higher and lower total weekly doses of IL-2 are partitioned into three equivalent doses that are administered according to a three-times-a-week dosing schedule, where the three equivalent doses are administered to the subject within a 7-day period, allowing for a minimum of 25 hours between doses and a maximum of 72 hours between doses.

The foregoing therapeutically effective doses of the reference IL-2 standard Proleukin®IL-2 are expressed in terms of MIU, which represent total amounts or absolute doses that are to be administered to a human subject on a weekly basis. The corresponding relative total weekly dose of Proleukin®IL-2 to be administered to a person to can readily be calculated. The average person is approximately 1.7 m². Thus, where the absolute total weekly dose of Proleukin®IL-2 to be administered is about 30.0 MIU to about 54.0 MIU, the corresponding relative total weekly dose of Proleukin®IL-2 is about 17.6 MIU/m² to about 31.8 MIU/m². Similarly, when the absolute total weekly dose is 30.0 MIU, 32.0 MIU, 35.0 MIU, 37.0 MIU, 40.0 MIU, 42.0 MIU, 45.0 MIU, 47.0 MIU, 50.0 MIU, 52.0 MIU, or 54.0 MIU, the corresponding relative total weekly dose is 17.6 MIU/m², 18.8 MIU/m², 20.6 MIU/m², 21.8 MIU/m², 23.5 MIU/m², 24.7 MIU/m², 26.5 MIU/m², 29.4 MIU/m², 30.6 MIU/m², and 31.8 MIU/m², respectively. These relative total weekly doses of IL-2 represent those doses that are to be administered in accordance with the constant IL-2 dosing regimen, and also represent the range of relative total weekly doses of IL-2 that are to be administered during the first time period of the two-level IL-2 dosing regimen. Those absolute total weekly doses that are to be administered during the second time period of the two-level IL-2 dosing regimen (i.e., within the range of about 18.0 MIU to about 39.0 MIU, including, for example, 18.0 MIU, 20.0 MIU, 23.0 MIU, 25.0 MIU, 27.0 MIU, 30.0 MIU, 32 MIU, 35.0 MIU, 37.0 MIU, and 39.0 MIU) have corresponding relative total weekly doses of about 10.6 MIU/m² to about 22.9 MIU/m², including 10.6 MIU/m², 11.8 MIU/m², 13.5 MIU/m², 14.7 MIU/m², 15.9 MIU/m², 17.6 MIU/m², 18.8 MIU/m², 20.6 MIU/m², 21.8 MIU/m², and 22.9 MIU/m², respectively.

MIU represents an international unit for a protein's biological activity. The international unit for IL-2 biological activity was established in 1988 by the World Health Organization (WHO) International Laboratory for Biological Standards. The IL-2 biological reference materials provided by the National Institute for Biological Standards and Control (NIBSC), which belongs to WHO, has 100 international units per ampoule of native human, Jurkat-derived IL-2. Activity of an IL-2 product can be measured against this international standard in an in vitro potency assay by HT-2 cell proliferation. Thus, for example, Proleukin®IL-2 has a biological activity of about 15 MIU per mg of this IL-2 product as determined by an HT-2 cell proliferation assay (see, for example, Gearing and Thorpe (1988) J. Immunological Methods 114:3-9; Nakanishi et al. (1984) J. Exp. Med. 160(6):1605-1621). The active moiety used in this product is the recombinant human IL-2 mutein aldesleukin (referred to as des-alanyl-1, serine-125 human interleukin-2; see U.S. Pat. No. 4,931,543). Using this information, one can calculate the recommended absolute total weekly dose of Proleukin®IL-2 in micrograms. Hence, where the absolute total weekly dose of Proleukin®IL-2 is about 30.0 MIU to about 54.0 MIU, the corresponding absolute total weekly dose of Proleukin®IL-2 in micrograms is about 2000 μg to about 3600 μg of this product. Similarly, where the absolute total weekly dose in MIU is about 18.0 MIU to about 39.0 MIU, the corresponding absolute total weekly dose in μg is about 1200 μg to about 2600 μg. Thus, given an absolute total weekly dose of Proleukin® IL-2 expressed in MIU, one of skill in the art can readily compute the corresponding relative total weekly dose expressed in MIU/m², or the absolute total weekly dose expressed in μg of this IL-2 product. See also Example 7 below.

For purposes of describing this invention, the doses of IL-2 have been presented using Proleukin®IL-2 as the reference IL-2 standard. One of skill in the art can readily determine what the corresponding doses would be for any IL-2 product comprising any form of IL-2 using a conversion factor based on comparative pharmacokinetic (PK) data and the serum concentration-time curve (AUC) for PK data collected during a 24-hour period for Proleukin®IL-2. Using PK data, the IL-2 exposure in human subjects that were administered a single dose of the reference IL-2 standard was determined. These subjects were selected such that they had not previously received exogenous IL-2 therapy (i.e., these subjects were naïve to IL-2 therapy). By “exogenous IL-2 therapy” is intended any intervention whereby a subject has been exposed to an exogenous source of IL-2, as opposed to exposure that occurs with the body's production of naturally occurring IL-2. Some of these subjects had received a single dose of 4.5 MIU of the reference IL-2 standard, while others had received a single dose of 7.5 or 18.0 MIU of the reference IL-2 standard. See Example 8 herein below.

Following administration of the single dose of the reference IL-2 standard, the IL-2 exposure in the blood serum was monitored over the first 10 to 12 hours post-injection, then extrapolated to 24 hours, and the resulting area under the serum concentration-time curve (AUC) for data collected during that 24-hour period was calculated. This area under the serum concentration-time curve is referred to herein as the AUC₀₋₂₄. Methods for measuring IL-2 exposure in this manner are well known in the art. See, for example, Gustavson (1998) J. Biol. Response Modifiers 1998:440-449; Thompson et al. (1987) Cancer Research 47:4202-4207; Kirchner et al. (1998) Br. J. Clin. Pharmacol. 46:5-10; Piscitelli et al. (1996) Pharmacotherapy 16(5):754-759; and Example 8 below. Thus, for those subjects receiving a dose of 4.5 MIU (300 μg) of Proleukin®IL-2, the AUC₀₋₂₄ value was 56 IU*hr/ml (SD=15); for those subjects receiving a dose of 7.5 MIU (500 μg) of Proleukin®IL-2, the AUC₀₋₂₄ value was 86 IU*hr/ml (SD=31.5); and for those subjects receiving the 18.0 MIU dose of Proleukin®IL-2, the AUC₀₋₂₄ value was 375 IU*hr/ml (SD=139). When such AUC₀₋₂₄ data is determined for the reference IL-2 standard, Proleukin® IL-2, the therapeutically effective doses described herein result in an IL-2 exposure within a range from about 22 IU*hour/ml serum to about 653 IU*hour/ml serum (see Example 8 below).

The sum of individual AUC₀₋₂₄ from individual doses will comprise the total weekly AUC₀₋₂₄ in partitioned individual doses. For example, if a dose of 18 MIU is administered three-times-a-week, the individual AUC₀₋₂₄ is estimated at 375 IU*hr/ml, and the total weekly AUC₀₋₂₄ will be 1125 IU*hr/ml based on linear assumption of increased AUC₀₋₂₄ with dose as shown in the Table 1 below.

TABLE 1
AUC_{0-24 values} obtained after administration of Proleukin ®IL-2.

| Proleukin ®IL-2 Dose | AUC₀₋₂₄ |
|---|---|
| (MIU/μg) | (IU*hr/ml) |
| 18/1200 | 375 |
| 30/2000 | 625 |
| 42/2800 | 875 |
| 54/3600 | 1125 |

The same total weekly AUC₀₋₂₄ of 1125 IU*hr/ml could also be obtained by dosing two-times-a-week at 27 MIU or dosing five-times-a-week at about 11 MIU.

For any other source of IL-2 (i.e., any other IL-2 formulation or any form of IL-2, including native or biologically active variant thereof such as muteins), a comparable recommended dose for use in the methods of the invention can be determined based on this AUC₀₋₂₄ data for Proleukin®IL-2. In this manner, a single dose of the IL-2 source of interest is administered to a human subject, and the level of IL-2 in the serum following this initial IL-2 exposure is determined by collecting PK data and generating an AUC₀₋₂₄ for the IL-2 source of interest. By “initial IL-2 exposure” is intended the subject used to measure IL-2 exposure has not previously undergone therapy with an exogenous source of IL-2 as noted above. This AUC₀₋₂₄ is then compared to the AUC₀₋₂₄ for Proleukin®IL-2 to determine a conversion factor that can be used to calculate a dose of the IL-2 source that is comparable to the recommended dose for Proleukin®IL-2. See, for example, the calculations for a representative monomeric IL-2 formulation, L2-7001, that are shown in Example 8 below. Thus, for any IL-2 source used in the methods of the present invention, the total weekly dose of IL-2 to be administered during a constant IL-2 dosing regimen, or during a two-level IL-2 dosing regimen, is in an amount equivalent to the recommended total weekly dose of the reference IL-2 standard, i.e., Proleukin®IL-2, as determined by the area under the serum concentration-time curve from human PK data.

The methods of the present invention also contemplate embodiments where a subject undergoing concurrent therapy with weekly administration of therapeutically effective doses of anti-CD20 antibody and administration of a two-level IL-2 dosing regimen is given a “drug holiday” or a time period off from IL-2 dosing, or from the IL-2 dosing and the anti-CD20 antibody dosing, between the conclusion of the first time period of the two-level IL-2 dosing regimen and the initiation of the second time period of the two-level IL-2 dosing regimen. In these embodiments, the two-level IL-2 dosing regimen is interrupted such that IL-2 dosing is withheld for a period of about 1 week to about 4 weeks following conclusion of the first time period of the two-level IL-2 dosing regimen during which the higher total weekly dose has been administered. During this time period off of IL-2 dosing, the subject can continue to receive weekly administration of a therapeutically effective dose of anti-CD20 antibody, or alternatively, the anti-CD20 antibody administration can also be stopped. The length of this interruption will depend upon the health of the subject, history of disease progression, and responsiveness of the subject to the initial IL-2/antibody therapy received during the first time period of the two-level IL-2 dosing regimen.

During this drug holiday (i.e., time period off of IL-2 administration, or time period off of IL-2 and anti-CD20 antibody administration), natural-killer (NK) cell counts are monitored to determine when the two-level IL-2 dosing regimen, or the two-level IL-2 dosing regimen and weekly administration of anti-CD20 antibody, are to be resumed. In this manner, NK cell counts are measured bi-weekly or monthly during the two-dose IL-2 dosing regimen, and at the conclusion of the first time period of the two-level IL-2 dosing regimen before the drug holiday is initiated. Where NK cell count exceeds an acceptable threshold level, the two-level IL-2 dosing regimen can be interrupted. By “acceptable threshold level” is intended the subject undergoing treatment has an NK cell count that is about 150 or greater, preferably 200 or greater. Following discontinuance of the IL-2 dosing, which may or may not include discontinuance of anti-CD20 antibody administration, NK cell counts are then measured once per week or twice per week thereafter, preferably once per week. An NK cell count falling below the acceptable threshold level of about 150, for example, an NK cell count of less than 150, is indicative of the necessity to resume the two-level IL-2 dosing regimen, or the two-level IL-2 dosing regimen and the anti-CD20 antibody dosing regimen where the drug holiday also includes time off of anti-CD20 antibody administration. Preferably the two-level IL-2 dosing regimen is resumed when NK cell count falls below a threshold level of about 200, i.e., an NK cell count of less than 200. At this time, the subject is administered the second time period of the two-level IL-2 dosing regimen, where lower total weekly doses of IL-2 are administered in combination with the weekly administration of therapeutically effective doses of the anti-CD20 antibody.

Where a subject undergoing therapy in accordance with the previously mentioned dosing regimens exhibits a partial response, or a relapse following a prolonged period of remission, subsequent courses of concurrent therapy may be needed to achieve complete remission of the disease. Thus, subsequent to a period of time off from a first treatment period, which may have comprised a constant IL-2 dosing regimen or a two-level IL-2 dosing regimen, a subject may receive one or more additional treatment periods comprising either constant or two-level IL dosing regimens in combination with anti-CD20 antibody administration. Such a period of time off between treatment periods is referred to herein as a time period of discontinuance. It is recognized that the length of the time period of discontinuance is dependent upon the degree of tumor response (i.e., complete versus partial) achieved with any prior treatment periods of concurrent therapy with these two therapeutic agents.

Thus, for example, where a subject is undergoing concurrent therapy with weekly doses of anti-CD20 antibody and a two-level IL-2 dosing regimen, which may or may not include a drug holiday between the first and second time periods of the two-level IL-2 dosing regimen, their treatment regimen may include multiple treatment sessions, each of which comprises concurrent therapy with weekly doses of anti-CD20 antibody and a two-level IL-2 dosing regimen. These multiple treatment sessions are referred to herein as maintenance cycles, where each maintenance cycle comprises anti-CD20 antibody administration in combination with a completed two-level IL-2 dosing regimen. By “completed two-level IL-2 dosing regimen” is intended the subject has been administered both the first period of higher total weekly dosing and the second period of lower total weekly dosing. The necessity for multiple maintenance cycles can be assessed by monitoring NK cell count in a manner similar to that used to determine when a drug holiday is warranted, and when such a drug holiday must be concluded. Thus, upon completion of the two-level IL-2 dosing regimen in any given maintenance cycle, the treating physician obtains a measurement of NK cell count. This indicator is then measured at monthly intervals (i.e., once a month) following completion of any given two-level IL-2 dosing regimen. As with drug holidays, an NK cell count falling below an acceptable threshold level (i.e., below about 150, preferably below about 200) is indicative of the need for administering another maintenance cycle to the subject. The duration between maintenance cycles can be about 1 month to about 6 months, including 1 month, 1.5 months, 2 months, 2.5 months, 3 months, 3.5 months, 4 months, 4.5 months, 5 months, 5.5 months, 6 months, or other such time periods falling within the range of about 1 month to about 6 months.

Thus, the administration methods of the present invention provide an improved means for managing non-Hodgkin's B-cell lymphomas in a human patient. Constant IL-2 dosing according to a twice-weekly or thrice-weekly dosing schedule provides an intermittent dosing schedule that allows for less frequent administration of the IL-2 during anti-CD20 antibody therapy, and better tolerability of long-term IL-2 therapy. The two-level IL-2 dosing regimen offers the opportunity to provide a patient with higher total weekly doses of IL-2, which provide for expansion of NK cell numbers that can be maintained by a lower dose during the subsequent weeks of IL-2 dosing. As IL-2 side effects are dose-related, the lowered dose will increase tolerability during the extended treatment period. This administration protocol has the additional attraction of providing drug holidays between the higher and lower total weekly dosing schedules, again contributing to increased tolerability of concurrent therapy with anti-CD20 antibody and IL-2.

The term “IL-2” as used herein refers to a lymphokine that is produced by normal peripheral blood lymphocytes and is present in the body at low concentrations. IL-2 was first described by Morgan et al. (1976) Science 193:1007-1008 and originally called T cell growth factor because of its ability to induce proliferation of stimulated T lymphocytes. It is a protein with a reported molecular weight in the range of 13,000 to 17,000 (Gillis and Watson (1980) J. Exp. Med. 159:1709) and has an isoelectric point in the range of 6-8.5. For purposes of the present invention, the term “IL-2” is intended to encompass any source of IL-2, including mammalian sources such as, e.g., mouse, rat, rabbit, primate, pig, and human, and may be native or obtained by recombinant techniques. The IL-2 may be the native polypeptide sequence, or can be a variant of the native IL-2 polypeptide as described herein below, so long as the variant IL-2 polypeptide retains the IL-2 biological activity of interest as defined herein. Preferably the IL-2 polypeptide or variant thereof is derived from a human source, and includes human IL-2 that is recombinantly produced, such as recombinant human IL-2 polypeptides produced by microbial hosts, and variants thereof that retain the IL-2 biological activity of interest. Any pharmaceutical composition comprising IL-2 as a therapeutically active component can be used to practice the present invention.

The IL-2 molecule useful in the methods of the invention may be a biologically active variant of native IL-2. Such variant IL-2 polypeptides should retain the desired biological activity of the native polypeptide such that the pharmaceutical composition comprising the variant polypeptide has the same therapeutic effect as the pharmaceutical composition comprising the native polypeptide when administered to a subject. That is, the variant polypeptide will serve as a therapeutically active component in the pharmaceutical composition in a manner similar to that observed for the native polypeptide. Methods are available in the art for determining whether a variant polypeptide retains the desired biological activity, and hence serves as a therapeutically active component in the pharmaceutical composition. Biological activity can be measured using assays specifically designed for measuring activity of the native polypeptide or protein, including assays described in the present invention. Additionally, antibodies raised against a biologically active native polypeptide can be tested for their ability to bind to the variant polypeptide, where effective binding is indicative of a polypeptide having a conformation similar to that of the native polypeptide.

For purposes of the present invention, the IL-2 biological activity of interest is the ability of IL-2 to activate and/or expand natural killer (NK) cells to mediate lymphokine activated killer (LAK) activity and antibody-dependent cellular cytotoxicity (ADCC). Thus, an IL-2 variant (for example, a mutein of human IL-2) for use in the methods of the present invention will activate and/or expand natural killer (NK) cells to mediate lymphokine activated killer (LAK) activity and antibody-dependent cellular cytotoxicity (ADCC). NK cells mediate spontaneous or natural cytotoxicity against certain cell targets referred to as “NK-cell sensitive” targets, such as the human erythroleukemia K562 cell line. Following activation by IL-2, NK cells acquire LAK activity. Such LAK activity can be assayed by the ability of IL-2 activated NK cells to kill a broad variety of tumor cells and other “NK-insensitive” targets, such as the Daudi B-cell lymphoma line, that are normally resistant to lysis by resting (nonactivated) NK cells. Similarly, ADCC activity can be assayed by the ability of IL-2 activated NK cells to lyse “NK-insensitive” target cells, such as Daudi B-cell lymphoma line, or other target cells not readily lysed by resting NK cells in the presence of optimal concentrations of relevant tumor cell specific antibodies. Methods for generating and measuring cytotoxic activity of NK/LAK cells and ADCC are known in the art. See for example, Current Protocols in Immunology: Immunologic Studies in Humans, Supplement 17, Unit 7.7, 7.18, and 7.27 (John Wiley & Sons, Inc., 1996). For purposes of the present invention, NK cells activated by an IL-2 variant for use in the methods of the present invention demonstrate a specific lysing activity of NK-insensitive cells in the presence (ADCC activity) or absence (LAK activity) of antibody, more particularly NK-insensitive Daudi cells in the presence of B-cell specific antibodies including rituximab, that is at least about 20% greater, or at least about 25%, or 30%, or 35%, or 40% greater than baseline lysing activity of resting NK cells (i..e., nonactivated) as measured using effector to target ratios between 12.5 to 50:1 in a standard 4-hr ⁵¹Cr-release cytotoxicity assay (see Current Protocols in Immunology: Immunologic Studies in Humans, Unit 7.7, Supplement 17, Section 17.18.1 (John Wiley & Sons, Inc., 1996). In some embodiments, the specific lysing activity of these IL-2 variant-activated NK cells is at least about 45% greater, at least about 50% greater, at least about 55% greater, or at least about 60% greater than baseline lysing activity of resting NK cells when measured as noted above.

Suitable biologically active variants of native or naturally occurring IL-2 can be fragments, analogues, and derivatives of that polypeptide. By “fragment” is intended a polypeptide consisting of only a part of the intact polypeptide sequence and structure, and can be a C-terminal deletion or N-terminal deletion of the native polypeptide. By “analogue” is intended an analogue of either the native polypeptide or of a fragment of the native polypeptide, where the analogue comprises a native polypeptide sequence and structure having one or more amino acid substitutions, insertions, or deletions. “Muteins”, such as those described herein, and peptides having one or more peptoids (peptide mimics) are also encompassed by the term analogue (see International Publication No. WO 91/04282). By “derivative” is intended any suitable modification of the native polypeptide of interest, of a fragment of the native polypeptide, or of their respective analogues, such as glycosylation, phosphorylation, polymer conjugation (such as with polyethylene glycol), or other addition of foreign moieties, so long as the desired biological activity of the native polypeptide is retained. Methods for making polypeptide fragments, analogues, and derivatives are generally available in the art.

For example, amino acid sequence variants of the polypeptide can be prepared by mutations in the cloned DNA sequence encoding the native polypeptide of interest. Methods for mutagenesis and nucleotide sequence alterations are well known in the art. See, for example, Walker and Gaastra, eds. (1983) Techniques in Molecular Biology (MacMillan Publishing Company, New York); Kunkel (1985) Proc. Natl. Acad. Sci. USA 82:488-492; Kunkel et al. (1987) Methods Enzymol. 154:367-382; Sambrook et al. (1989) Molecular Cloning: A Laboratory Manual (Cold Spring Harbor, N.Y.); U.S. Pat. No. 4,873,192; and the references cited therein. Guidance as to appropriate amino acid substitutions that do not affect biological activity of the polypeptide of interest may be found in the model of Dayhoff et al. (1978) in Atlas of Protein Sequence and Structure (Natl. Biomed. Res. Found., Washington, D.C.). Conservative substitutions, such as exchanging one amino acid with another having similar properties, may be preferred. Examples of conservative substitutions include, but are not limited to, Gly [symbol] Ala, Val [symbol] Ile [symbol] Leu, Asp [symbol] Glu, Lys [symbol] Arg, Asn [symbol] Gln, and Phe [symbol] Trp [symbol] Tyr.

In constructing variants of the IL-2 polypeptide of interest, modifications are made such that variants continue to possess the desired activity. Obviously, any mutations made in the DNA encoding the variant polypeptide must not place the sequence out of reading frame and preferably will not create complementary regions that could produce secondary mRNA structure. See EP Patent Application Publication No. 75,444.

Biologically active variants of IL-2 will generally have at least 70%, preferably at least 80%, more preferably about 90% to 95% or more, and most preferably about 98% or more amino acid sequence identity to the amino acid sequence of the reference polypeptide molecule, which serves as the basis for comparison. Thus, where the IL-2 reference molecule is human IL-2, a biologically active variant thereof will have at least 70%, preferably at least 80%, more preferably about 90% to 95% or more, and most preferably about 98% or more sequence identity to the amino acid sequence for human IL-2. A biologically active variant of a native polypeptide of interest may differ from the native polypeptide by as few as 1-15 amino acids, as few as 1-10, such as 6-10, as few as 5, as few as 4, 3, 2, or even 1 amino acid residue. By “sequence identity” is intended the same amino acid residues are found within the variant polypeptide and the polypeptide molecule that serves as a reference when a specified, contiguous segment of the amino acid sequence of the variants is aligned and compared to the amino acid sequence of the reference molecule. The percentage sequence identity between two amino acid sequences is calculated by determining the number of positions at which the identical amino acid residue occurs in both sequences to yield the number of matched positions, dividing the number of matched positions by the total number of positions in the segment undergoing comparison to the reference molecule, and multiplying the result by 100 to yield the percentage of sequence identity.

For purposes of optimal alignment of the two sequences, the contiguous segment of the amino acid sequence of the variants may have additional amino acid residues or deleted amino acid residues with respect to the amino acid sequence of the reference molecule. The contiguous segment used for comparison to the reference amino acid sequence will comprise at least twenty (20) contiguous amino acid residues, and may be 30, 40, 50, 100, or more residues. Corrections for increased sequence identity associated with inclusion of gaps in the variants' amino acid sequence can be made by assigning gap penalties. Methods of sequence alignment are well known in the art for both amino acid sequences and for the nucleotide sequences encoding amino acid sequences.

Thus, the determination of percent identity between any two sequences can be accomplished using a mathematical algorithm. One preferred, non-limiting example of a mathematical algorithm utilized for the comparison of sequences is the algorithm of Myers and Miller (1988) CABIOS4:11-17. Such an algorithm is utilized in the ALIGN program (version 2.0), which is part of the GCG sequence alignment software package. A PAM120 weight residue table, a gap length penalty of 12, and a gap penalty of 4 can be used with the ALIGN program when comparing amino acid sequences. Another preferred, nonlimiting example of a mathematical algorithm for use in comparing two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:403. BLAST nucleotide searches can be performed with the NBLAST program, score=100, wordlength=12, to obtain nucleotide sequences homologous to a nucleotide sequence encoding the polypeptide of interest. BLAST protein searches can be performed with the XBLAST program, score=50, wordlength=3, to obtain amino acid sequences homologous to the polypeptide of interest. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST) can be used. See http://www.ncbi.nlm.nih.gov. Also see the ALIGN program (Dayhoff (1978) in Atlas of Protein Sequence and Structure 5:Suppl. 3 (National Biomedical Research Foundation, Washington, D.C.) and programs in the Wisconsin Sequence Analysis Package, Version 8 (available from Genetics Computer Group, Madison, Wis.), for example, the GAP program, where default parameters of the programs are utilized.

When considering percentage of amino acid sequence identity, some amino acid residue positions may differ as a result of conservative amino acid substitutions, which do not affect properties of protein function. In these instances, percent sequence identity may be adjusted upwards to account for the similarity in conservatively substituted amino acids. Such adjustments are well known in the art. See, for example, Myers and Miller (1988) Computer Applic. Biol. Sci. 4:11-17.

The precise chemical structure of a polypeptide having IL-2 activity depends on a number of factors. As ionizable amino and carboxyl groups are present in the molecule, a particular polypeptide may be obtained as an acidic or basic salt, or in neutral form. All such preparations that retain their biological activity when placed in suitable environmental conditions are included in the definition of polypeptides having IL-2 activity as used herein. Further, the primary amino acid sequence of the polypeptide may be augmented by derivatization using sugar moieties (glycosylation) or by other supplementary molecules such as lipids, phosphate, acetyl groups and the like. It may also be augmented by conjugation with saccharides. Certain aspects of such augmentation are accomplished through post-translational processing systems of the producing host; other such modifications may be introduced in vitro. In any event, such modifications are included in the definition of an IL-2 polypeptide used herein so long as the IL-2 activity of the polypeptide is not destroyed. It is expected that such modifications may quantitatively or qualitatively affect the activity, either by enhancing or diminishing the activity of the polypeptide, in the various assays. Further, individual amino acid residues in the chain may be modified by oxidation, reduction, or other derivatization, and the polypeptide may be cleaved to obtain fragments that retain activity. Such alterations that do not destroy activity do not remove the polypeptide sequence from the definition of IL-2 polypeptides of interest as used herein.

The art provides substantial guidance regarding the preparation and use of polypeptide variants. In preparing the IL-2 variants, one of skill in the art can readily determine which modifications to the native protein nucleotide or amino acid sequence will result in a variant that is suitable for use as a therapeutically active component of a pharmaceutical composition used in the methods of the present invention.

The IL-2 for use in the methods of the present invention may be from any source, but preferably is recombinant IL-2. By “recombinant IL-2” is intended interleukin-2 that has comparable biological activity to native-sequence IL-2 and that has been prepared by recombinant DNA techniques as described, for example, by Taniguchi et al. (1983) Nature 302:305-310 and Devos (1983) Nucleic Acids Research 11:4307-4323 or mutationally altered IL-2 as described by Wang et al. (1984) Science 224:1431-1433. In general, the gene coding for IL-2 is cloned and then expressed in transformed organisms, preferably a microorganism, for example E. coli, as described herein. The host organism expresses the foreign gene to produce IL-2 under expression conditions. Synthetic recombinant IL-2 can also be made in eukaryotes, such as yeast or human cells. Processes for growing, harvesting, disrupting, or extracting the IL-2 from cells are known in the art as evidenced by, for example, U.S. Pat. Nos. 4,604,377; 4,738,927; 4,656,132; 4,569,790; 4,748,234; 4,530,787; 4,572,798; 4,748,234; and 4,931,543.

For examples of variant IL-2 proteins, see European Patent (EP) Publication No. EP 136,489 (which discloses one or more of the following alterations in the amino acid sequence of naturally occurring IL-2: Asn26 to Gln26; Trp121 to Phe121; Cys58 to Ser58 or Ala58, Cys105 to Ser105 or Ala105; Cys125 to Ser125 or Ala125; deletion of all residues following Arg 120; and the Met-1 forms thereof); and the recombinant IL-2 muteins described in European Patent Application No. 83306221.9, filed Oct. 13, 1983 (published May 30, 1984 under Publication No. EP 109,748), which is the equivalent to Belgian Patent No. 893,016, and commonly owned U.S. Pat. No. 4,518,584 (which disclose recombinant human IL-2 mutein wherein the cysteine at position 125, numbered in accordance with native human IL-2, is deleted or replaced by a neutral amino acid; alanyl-ser125-IL-2; and des-alanayl-ser125-IL-2). See also U.S. Pat. No. 4,752,585 (which discloses the following variant IL-2 proteins: ala104 ser125 IL-2, ala104 IL-2, ala104 ala125 IL-2, val104 ser125 IL-2, val104 IL-2, val104 ala125 IL-2, des-ala1 ala104 ser125 IL-2, des-ala1 ala104 IL-2, des-ala1 ala104 ala125 IL-2, des-ala1 val104 ser125 IL-2, des-ala1 val104 IL-2, des-ala1 val104 ala125 IL-2, des-ala1 des-pro2 ala104 ser125 IL-2, des-ala1 des-pro2 ala104 IL-2, des-ala1 des-pro2 ala104 ala125 IL-2, des-ala1 des-pro2 val104 ser125 IL-2, des-ala1 des-pro2 val104 IL-2, des-ala1 des-pro2 val104 ala125 IL-2, des-ala1 des-pro2 des-thr3 ala104 ser125 IL-2, des-ala1 des-pro2 des-thr3 ala104 IL-2, des-ala1 des-pro2 des-thr3 ala104 ala125 IL-2, des-ala1 des-pro2 des-thr3 val104 ser125 IL-2, des-ala1 des-pro2 des-thr3 val104 IL-2, des-ala1 des-pro2 des-thr3 val104 ala125 IL-2, des-ala1 des-pro2 des-thr3 des-ser4 ala104 ser125 IL-2, des-ala1 des-pro2 des-thr3 des-ser4 ala104 IL-2, des-ala1 des-pro2 des-thr3 des-ser4 ala104 ala125 IL-2, des-ala1 des-pro2 des-thr3 des-ser4 val104 ser125 IL-2, des-ala1 des-pro2 des-thr3 des-ser4 val104 IL-2, des-ala1 des-pro2 des-thr3 des-ser4 val104 ala125 IL-2, des-ala1 des-pro2 des-thr3 des-ser4 des-ser5 ala104 ser125 IL-2, des-ala1 des-pro2 des-thr3 des-ser4 des-ser5 ala104 IL-2, des-ala1 des-pro2 des-thr3 des-ser4 des-ser5 ala104 ala125 IL-2, des-ala1 des-pro2 des-thr3 des-ser4 des-ser5 val104 ser125 IL-2, des-ala1 des-pro2 des-thr3 des-ser4 des-ser5 val 104 IL-2, des-ala1 des-pro2 des-thr3 des-ser4 des-ser5 val104 ala125 IL-2, des-ala1 des-pro2 des-thr3 des-ser4 des-ser5 des-ser6 ala104 ala125 IL-2, des-ala1 des-pro2 des-thr3 des-ser4 des-ser5 des-ser6 ala104 IL-2, des-ala1 des-pro2 des-thr3 des-ser4 des-ser5 des-ser6 ala104 ser125 IL-2, des-ala1 des-pro2 des-thr3 des-ser4 des-ser5 des-ser6 val104 ser125 IL-2, des-ala1 des-pro2 des-thr3 des-ser4 des-ser5 des-ser6 val104 IL-2, and des-ala1 des-pro2 des-thr3 des-ser4 des-ser5 des-ser6 val104 ala125 IL-2) and U.S. Pat. No. 4,931,543 (which discloses the IL-2 mutein des-alanyl-1, serine-125 human IL-2 used in the examples herein, as well as the other IL-2 muteins).

Also see European Patent Publication No. EP 200,280 (published Dec. 10, 1986), which discloses recombinant IL-2 muteins wherein the methionine at position 104 has been replaced by a conservative amino acid. Examples include the following muteins: ser4 des-ser5 ala104 IL-2; des-ala1 des-pro2 des-thr3 des-ser4 des-ser5 ala104 ala125 IL-2; des-ala1 des-pro2 des-thr3 des-ser4 des-ser5 glu104 ser125 IL-2; des-ala1 des-pro2 des-thr3 des-ser4 des-ser5 glu104 IL-2; des-ala1 des-pro2 des-thr3 des-ser4 des-ser5 glu104 ala125 IL-2; des-ala1 des-pro2 des-thr3 des-ser4 des-ser5 des-ser6 ala104 ala125 IL-2; des-ala1 des-pro2 des-thr3 des-ser4 des-ser5 des-ser6 ala104 IL-2; des-ala1 des-pro2 des-thr3 des-ser4 des-ser5 des-ser6 ala104 ser125 IL-2; des-ala1 des-pro2 des-thr3 des-ser4 des-ser5 des-ser6 glu104 ser125 IL-2; des-ala1 des-pro2 des-thr3 des-ser4 des-ser5 des-ser6 glu104 IL-2; and des-ala1 des-pro2 des-thr3 des-ser4 des-ser5 des-ser6 glu104 ala125 IL-2. See also European Patent Publication No. EP 118,617 and U.S. Pat. No. 5,700,913, which disclose unglycosylated human IL-2 variants bearing alanine instead of native IL-2's methionine as the N-terminal amino acid; an unglycosylated human IL-2 with the initial methionine deleted such that proline is the N-terminal amino acid; and an unglycosylated human IL-2 with an alanine inserted between the N-terminal methionine and proline amino acids.

Other IL-2 muteins include the those disclosed in WO 99/60128 (substitutions of the aspartate at position 20 with histidine or isoleucine, the asparagine at position 88 with arginine, glycine, or isoleucine, or the glutamine at position 126 with leucine or gulatamic acid), which reportedly have selective activity for high affinity IL-2 receptors expressed by cells expressing T cell receptors in preference to NK cells and reduced IL-2 toxicity; the muteins disclosed in U.S. Pat. No. 5,229,109 (substitutions of arginine at position 38 with alanine, or substitutions of phenylalanine at position 42 with lysine), which exhibit reduced binding to the high affinity IL-2 receptor when compared to native IL-2 while maintaining the ability to stimulate LAK cells; the muteins disclosed in International Publication No. WO 00/58456 (altering or deleting a naturally occurring (x)D(y) sequence in native IL-2 where D is aspartic acid, (x) is leucine, isoleucine, glycine, or valine, and (y) is valine, leucine or serine), which are claimed to reduce vascular leak syndrome; the IL-2 p1-30 peptide disclosed in International Publication No. WO 00/04048 (corresponding to the first 30 amino acids of IL-2, which contains the entire a-helix A of IL-2 and interacts with the b chain of the IL-2 receptor), which reportedly stimulates NK cells and induction of LAK cells; and a mutant form of the IL-2 p1-30 peptide also disclosed in WO 00/04048 (substitution of aspartic acid at position 20 with lysine), which reportedly is unable to induce vascular bleeds but remains capable of generating LAK cells. Additionally, IL-2 can be modified with polyethylene glycol to provide enhanced solubility and an altered pharmokinetic profile (see U.S. Pat. No. 4,766,106).

The term IL-2 as used herein is also intended to include IL-2 fusions or conjugates comprising IL-2 fused to a second protein or covalently conjugated to polyproline or a water-soluble polymer to reduce dosing frequencies or to improve IL-2 tolerability. For example, the IL-2 (or a variant thereof as defined herein) can be fused to human albumin or an albumin fragment using methods known in the art (see WO 01/79258). Alternatively, the IL-2 can be covalently conjugated to polyproline or polyethylene glycol homopolymers and polyoxyethylated polyols, wherein the homopolymer is unsubstituted or substituted at one end with an alkyl group and the poplyol is unsubstituted, using methods known in the art (see, for example, U.S. Pat. Nos. 4,766,106, 5,206,344, and 4,894,226).

Any pharmaceutical composition comprising IL-2 as the therapeutically active component can be used in the methods of the invention. Such pharmaceutical compositions are known in the art and include, but are not limited to, those disclosed in U.S. Pat. Nos. 4,745,180; 4,766,106; 4,816,440; 4,894,226; 4,931,544; and 5,078,997. Thus liquid, lyophilized, or spray-dried compositions comprising IL-2 that are known in the art may be prepared as an aqueous or nonaqueous solution or suspension for subsequent administration to a subject in accordance with the methods of the invention. Each of these compositions will comprise IL-2 as a therapeutically or prophylactically active component. By “therapeutically or prophylactically active component” is intended the IL-2 is specifically incorporated into the composition to bring about a desired therapeutic or prophylactic response with regard to treatment, prevention, or diagnosis of a disease or condition within a subject when the pharmaceutical composition is administered to that subject. Preferably the pharmaceutical compositions comprise appropriate stabilizing agents, bulking agents, or both to minimize problems associated with loss of protein stability and biological activity during preparation and storage.

In preferred embodiments of the invention, the IL-2 containing pharmaceutical compositions useful in the methods of the invention are compositions comprising stabilized monomeric IL-2, compositions comprising multimeric IL-2, and compositions comprising stabilized lyophilized or spray-dried IL-2.

Pharmaceutical compositions comprising stabilized monomeric IL-2 are disclosed in International Publication No. WO 01/24814, entitled “Stabilized Liquid Polypeptide-Containing Pharmaceutical Compositions.” By “monomeric” IL-2 is intended the protein molecules are present substantially in their monomer form, not in an aggregated form, in the pharmaceutical compositions described herein. Hence covalent or hydrophobic oligomers or aggregates of IL-2 are not present. Briefly, the IL-2 in these liquid compositions is formulated with an amount of an amino acid base sufficient to decrease aggregate formation of IL-2 during storage. The amino acid base is an amino acid or a combination of amino acids, where any given amino acid is present either in its free base form or in its salt form. Preferred amino acids are selected from the group consisting of arginine, lysine, aspartic acid, and glutamic acid. These compositions further comprise a buffering agent to maintain pH of the liquid compositions within an acceptable range for stability of IL-2, where the buffering agent is an acid substantially free of its salt form, an acid in its salt form, or a mixture of an acid and its salt form. Preferably the acid is selected from the group consisting of succinic acid, citric acid, phosphoric acid, and glutamic acid. Such compositions are referred to herein as stabilized monomeric IL-2 pharmaceutical compositions.

The amino acid base in these compositions serves to stabilize the IL-2 against aggregate formation during storage of the liquid pharmaceutical composition, while use of an acid substantially free of its salt form, an acid in its salt form, or a mixture of an acid and its salt form as the buffering agent results in a liquid composition having an osmolarity that is nearly isotonic. The liquid pharmaceutical composition may additionally incorporate other stabilizing agents, more particularly methionine, a nonionic surfactant such as polysorbate 80, and EDTA, to further increase stability of the polypeptide. Such liquid pharmaceutical compositions are said to be stabilized, as addition of amino acid base in combination with an acid substantially free of its salt form, an acid in its salt form, or a mixture of an acid and its salt form, results in the compositions having increased storage stability relative to liquid pharmaceutical compositions formulated in the absence of the combination of these two components.

These liquid pharmaceutical compositions comprising stabilized monomeric IL-2 may either be used in an aqueous liquid form, or stored for later use in a frozen state, or in a dried form for later reconstitution into a liquid form or other form suitable for administration to a subject in accordance with the methods of present invention. By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53).

Other examples of IL-2 formulations that comprise IL-2 in its nonaggregated monomeric state include those described in Whittington and Faulds (1993) Drugs 46(3):446-514. These formulations include the recombinant IL-2 product in which the recombinant IL-2 mutein Teceleukin (unglycosylated human IL-2 with a methionine residue added at the amino-terminal) is formulated with 0.25% human serum albumin in a lyophilized powder that is reconstituted in isotonic saline, and the recombinant IL-2 mutein Bioleukin (human IL-2 with a methionine residue added at the amino-terminal, and a substitution of the cysteine residue at position 125 of the human IL-2 sequence with alanine) formulated such that 0.1 to 1.0 mg/ml IL-2 mutein is combined with acid, wherein the formulation has a pH of 3.0 to 4.0, advantageously no buffer, and a conductivity of less than 1000 mmhos/cm (advantageously less than 500 mmhos/cm). See EP 373,679; Xhang et al. (1996) Pharmaceut. Res. 13(4):643-644; and Prestrelski et al. (1995) Pharmaceut. Res. 12(9):1250-1258.

Examples of pharmaceutical compositions comprising multimeric IL-2 are disclosed in commonly owned U.S. Pat. No. 4,604,377. By “multimeric” is intended the protein molecules are present in the pharmaceutical composition in a microaggregated form having an average molecular association of 10-50 molecules. These multimers are present as loosely bound, physically associated IL-2 molecules. A lyophilized form of these compositions is available commercially under the tradename Proleukin® IL-2 (Chiron Corporation). The lyophilized formulations disclosed in this reference comprise selectively oxidized, microbially produced recombinant IL-2 in which the recombinant IL-2 is admixed with a water soluble carrier such as mannitol that provides bulk, and a sufficient amount of sodium dodecyl sulfate to ensure the solubility of the recombinant IL-2 in water. These compositions are suitable for reconstitution in aqueous injections for parenteral administration and are stable and well tolerated in human patients. When reconstituted, the IL-2 retains its multimeric state. Such lyophilized or liquid compositions comprising multimeric IL-2 are encompassed by the methods of the present invention. Such compositions are referred to herein as multimeric IL-2 pharmaceutical compositions.

The methods of the present invention may also use stabilized lyophilized or spray-dried pharmaceutical compositions comprising IL-2, which may be reconstituted into a liquid or other suitable form for administration in accordance with methods of the invention. Such pharmaceutical compositions are disclosed in International Publication No. WO 01/24814, entitled “Methods for Pulmonary Delivery of Interleukin-2.” These compositions may further comprise at least one bulking agent, at least one agent in an amount sufficient to stabilize the protein during the drying process, or both. By “stabilized” is intended the IL-2 protein or variants thereof retains its monomeric or multimeric form as well as its other key properties of quality, purity, and potency following lyophilization or spray-drying to obtain the solid or dry powder form of the composition. In these compositions, preferred carrier materials for use as a bulking agent include glycine, mannitol, alanine, valine, or any combination thereof, most preferably glycine. The bulking agent is present in the formulation in the range of 0% to about 10% (w/v), depending upon the agent used. Preferred carrier materials for use as a stabilizing agent include any sugar or sugar alcohol or any amino acid. Preferred sugars include sucrose, trehalose, raffinose, stachyose, sorbitol, glucose, lactose, dextrose or any combination thereof, preferably sucrose. When the stabilizing agent is a sugar, it is present in the range of about 0% to about 9.0% (w/v), preferably about 0.5% to about 5.0%, more preferably about 1.0% to about 3.0%, most preferably about 1.0%. When the stabilizing agent is an amino acid, it is present in the range of about 0% to about 1.0% (w/v), preferably about 0.3% to about 0.7%, most preferably about 0.5%. These stabilized lyophilized or spray-dried compositions may optionally comprise methionine, ethylenediaminetetracetic acid (EDTA) or one of its salts such as disodium EDTA or other chelating agent, which protect the IL-2 against methionine oxidation. Use of these agents in this manner is described in International Publication No. WO 01/24814. The stabilized lyophilized or spray-dried compositions may be formulated using a buffering agent, which maintains the pH of the pharmaceutical composition within an acceptable range, preferably between about pH 4.0 to about pH 8.5, when in a liquid phase, such as during the formulation process or following reconstitution of the dried form of the composition. Buffers are chosen such that they are compatible with the drying process and do not affect the quality, purity, potency, and stability of the protein during processing and upon storage.

The previously described stabilized monomeric, multimeric, and stabilized lyophilized or spray-dried IL-2 pharmaceutical compositions represent suitable compositions for use in the methods of the invention. However, any pharmaceutical composition comprising IL-2 as a therapeutically active component is encompassed by the methods of the invention.

As used herein, the term “anti-CD20 antibody” encompasses any antibody that specifically recognizes the CD20 B-cell surface antigen, including polyclonal anti-CD20 antibodies, monoclonal anti-CD20 antibodies, human anti-CD20 antibodies, humanized anti-CD20 antibodies, chimeric anti-CD20 antibodies, xenogeneic anti-CD20 antibodies, and fragments of these anti-CD20 antibodies that specifically recognize the CD20 B-cell surface antigen. Preferably the antibody is monoclonal in nature. By “monoclonal antibody” is intended an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site, i.e., the CD20 B-cell surface antigen in the present invention. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256:495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352:624-628 and Marks et al. (1991) J. Mol. Biol. 222:581-597, for example.

Anti-CD20 antibodies of murine origin are suitable for use in the methods of the present invention. Examples of such murine anti-CD20 antibodies include, but are not limited to, the B1 antibody (described in U.S. Pat. No. 6,015,542); the IF5 antibody (see Press et al. (1989) J. Clin. Oncol. 7:1027); NKI-B20 and BCA-B20 anti-CD20 antibodies (described in Hooijberg et al. (1995) Cancer Research 55:840-846); and IDEC-2B8 (available commercially from IDEC Pharmaceuticals Corp., San Diego, Calif.); the 2H7 antibody (described in Clark et al. (1985) Proc. Natl. Acad. Sci. USA 82:1766-1770; and others described in Clark et al. (1985) supra and Stashenko et al. (1980) J. Immunol. 125:1678-1685.

The term “anti-CD20 antibody” as used herein encompasses chimeric anti-CD20 antibodies. By “chimeric antibodies” is intended antibodies that are most preferably derived using recombinant deoxyribonucleic acid techniques and which comprise both human (including immunologically “related” species, e.g., chimpanzee) and non-human components. Thus, the constant region of the chimeric antibody is most preferably substantially identical to the constant region of a natural human antibody; the variable region of the chimeric antibody is most preferably derived from a non-human source and has the desired antigenic specificity to the CD20 cell surface antigen. The non-human source can be any vertebrate source that can be used to generate antibodies to a human CD20 cell surface antigen or material comprising a human CD20 cell surface antigen. Such non-human sources include, but are not limited to, rodents (e.g., rabbit, rat, mouse, etc.; see, for example, U.S. Pat. No. 4,816,567) and non-human primates (e.g., Old World Monkey, Ape, etc.; see, for example, U.S. Pat. Nos. 5,750,105 and 5,756,096). Most preferably, the non-human component (variable region) is derived from a murine source. As used herein, the phrase “immunologically active” when used in reference to chimeric anti-CD20 antibodies means a chimeric antibody that binds human C1q, mediates complement dependent lysis (“CDC”) of human B lymphoid cell lines, and lyses human target cells through antibody dependent cellular cytotoxicity (“ADCC”). Examples of chimeric anti-CD20 antibodies include, but are not limited to, IDEC-C2B8, available commercially under the name rituximab (IDEC Pharmaceuticals Corp., San Diego, Calif.) and described in U.S. Pat. Nos. 5,736,137, 5,776,456, and 5,843,439; the chimeric antibodies described in U.S. Pat. No. 5,750,105; those described in U.S. Pat. Nos. 5,500,362; 5,677,180; 5,721,108; and 5,843,685.

Humanized anti-CD20 antibodies are also encompassed by the term anti-CD20 antibody as used herein. By “humanized” is intended forms of anti-CD20 antibodies that contain minimal sequence derived from non-human immunoglobulin sequences. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. See, for example, U.S. Pat. Nos. 5,225,539; 5,585,089; 5,693,761; 5,693,762; 5,859,205. In some instances, framework residues of the human immunoglobulin are replaced by corresponding non-human residues (see, for example, U.S. Pat. Nos. 5,585,089; 5,693,761; 5,693,762). Furthermore, humanized antibodies may comprise residues that are not found in the recipient antibody or in the donor antibody. These modifications are made to further refine antibody performance (e.g., to obtain desired affinity). In general, the humanized antibody will comprise substantially all of at least one, and typically two, variable domains, in which all or substantially all of the hypervariable regions correspond to those of a non-human immunoglobulin and all or substantially all of the framework regions are those of a human immunoglobulin sequence. The humanized antibody optionally also will comprise at least a portion of an immunoglobulin constant region (Fc), typically that of a human immunoglobulin. For further details see Jones et al. (1986) Nature 331:522-525; Riechmann et al. (1988) Nature 332:323-329; and Presta (1992) Curr. Op. Struct. Biol. 2:593-596.

Also encompassed by the term anti-CD20 antibodies are xenogeneic or modified anti-CD20 antibodies produced in a non-human mammalian host, more particularly a transgenic mouse, characterized by inactivated endogenous immunoglobulin (Ig) loci. In such transgenic animals, competent endogenous genes for the expression of light and heavy subunits of host immunoglobulins are rendered non-functional and substituted with the analogous human immunoglobulin loci. These transgenic animals produce human antibodies in the substantial absence of light or heavy host immunoglobulin subunits. See, for example, U.S. Pat. No. 5,939,598.

Fragments of the anti-CD20 antibodies are suitable for use in the methods of the invention so long as they retain the desired affinity of the full-length antibody. Thus, a fragment of an anti-CD20 antibody will retain the ability to bind to the CD20 B-cell surface antigen. Fragments of an antibody comprise a portion of a full-length antibody, generally the antigen binding or variable region thereof. Examples of antibody fragments include, but are not limited to, Fab, Fab′, F(ab′)₂, and Fv fragments and single-chain antibody molecules. By “single-chain Fv” or “sFv” antibody fragments is intended fragments comprising the V_{H} and V_{L} domains of an antibody, wherein these domains are present in a single polypeptide chain. See, for example, U.S. Pat. Nos. 4,946,778; 5,260,203; 5,455,030; 5,856,456. Generally, the Fv polypeptide further comprises a polypeptide linker between the V_{H} and V_{L} domains that enables the sFv to form the desired structure for antigen binding. For a review of sFv see Pluckthun (1994) in The Pharmacology of Monoclonal Antibodies, Vol. 113, ed. Rosenburg and Moore (Springer-Verlag, New York), pp. 269-315.

Antibodies or antibody fragments can be isolated from antibody phage libraries generated using the techniques described in McCafferty et al. (1990) Nature 348:552-554 (1990). Clackson et al. (1991) Nature 352:624-628 and Marks et al. (1991) J. Mol. Biol. 222:581-597 describe the isolation of murine and human antibodies, respectively, using phage libraries. Subsequent publications describe the production of high affinity (nM range) human antibodies by chain shuffling (Marks et al. (1992) Bio/Technology 10:779-783), as well as combinatorial infection and in vivo recombination as a strategy for constructing very large phage libraries (Waterhouse et al. (1993) Nucleic. Acids Res. 21:2265-2266). Thus, these techniques are viable alternatives to traditional monoclonal antibody hybridoma techniques for isolation of monoclonal antibodies.

A humanized antibody has one or more amino acid residues introduced into it from a source that is non-human. These non-human amino acid residues are often referred to as “donor” residues, which are typically taken from a “donor” variable domain. Humanization can be essentially performed following the method of Winter and co-workers (Jones et al. (1986) Nature 321:522-525; Riechmann et al. (1988) Nature 332:323-327; Verhoeyen et al. (1988) Science 239:1534-1536), by substituting rodent CDRs or CDR sequences for the corresponding sequences of a human antibody. See, for example, U.S. Pat. Nos. 5,225,539; 5,585,089; 5,693,761; 5,693,762; 5,859,205. Accordingly, such “humanized” antibodies may include antibodies wherein substantially less than an intact human variable domain has been substituted by the corresponding sequence from a non-human species. In practice, humanized antibodies are typically human antibodies in which some CDR residues and possibly some framework residues are substituted by residues from analogous sites in rodent antibodies. See, for example, U.S. Pat. Nos. 5,225,539; 5,585,089; 5,693,761; 5,693,762; 5,859,205. See also U.S. Pat. No. 6,180,370, and International Publication No. WO 01/27160, where humanized antibodies and techniques for producing humanized antibodies having improved affinity for a predetermined antigen are disclosed.

Various techniques have been developed for the production of antibody fragments. Traditionally, these fragments were derived via proteolytic digestion of intact antibodies (see, e.g., Morimoto et al. (1992) Journal of Biochemical and Biophysical Methods 24:107-117 (1992) and Brennan et al. (1985) Science 229:81). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al. (1992) Bio/Technology 10:163-167). According to another approach, F(ab′)₂ fragments can be isolated directly from recombinant host cell culture. Other techniques for the production of antibody fragments will be apparent to the skilled practitioner.

Further, any of the previously described anti-CD20 antibodies may be conjugated prior to use in the methods of the present invention. Such conjugated antibodies are available in the art. Thus, the anti-CD20 antibody may be labeled using an indirect labeling or indirect labeling approach. By “indirect labeling” or “indirect labeling approach” is intended that a chelating agent is covalently attached to an antibody and at least one radionuclide is inserted into the chelating agent. See, for example, the chelating agents and radionuclides described in Srivagtava and Mease (1991) Nucl. Med. Bio. 18: 589-603. Alternatively, the anti-CD20 antibody may be labeled using “direct labeling” or a “direct labeling approach”, where a radionuclide is covalently attached directly to an antibody (typically via an amino acid residue). Preferred radionuclides are provided in Srivagtava and Mease (1991) supra. The indirect labeling approach is particularly preferred. See also, for example, labeled forms of anti-CD20 antibodies described in U.S. Pat. No. 6,015,542.

The anti-CD20 antibodies are typically provided by standard technique within a pharmaceutically acceptable buffer, for example, sterile saline, sterile buffered water, propylene glycol, combinations of the foregoing, etc. Methods for preparing parentally administerable agents are described in Remington's Pharmaceutical Sciences (18^{th} ed.; Mack Pub. Co.: Eaton, Pa. 1990). See also, for example, International Publication No. WO 98/56418, which describes stabilized antibody pharmaceutical formulations suitable for use in the methods of the present invention.

The present invention further provides a method for predicting clinical response of a subject undergoing a time period of concurrent therapy with anti-CD20 antibody and IL-2 in accordance with the dosing regimens disclosed herein. The method comprises monitoring natural killer (NK) cell expansion in said subject at about 1 week to about 7 weeks post-initiation, preferably at about 1 to about 14 weeks post-initiation of the time period of concurrent therapy with these two therapeutic agents. Preferably NK cell counts are determined prior to the start of concurrent therapy, and are monitored throughout the time period of concurrent therapy so that the time course of NK cell expansion can be followed. In this manner, NK cell counts are determined weekly in a patient over the course of concurrent therapy with anti-CD20 antibody and IL-2 and for a period of 4-6 weeks following the final IL-2 administration. Methods for determining NK cell counts are known in the art. See, for example, methods disclosed in Suzuki et al. ((1983) J. Immunol. 130:981-987; Herberman (1987) Prog. Clin. Biol. Res. 244:267-274; and Meropol et al. (1998) Cancer Immunol. Immunother. 46:318-326. When undergoing combination therapy with IL-2, and anti-CD20 antibody as outlined herein with twice-weekly, thrice-weekly, or daily IL-2 dosing for 4 weeks, those patients having expansion of NK cell counts to greater than about 200 cells/μl at 10 weeks post-initiation of therapy are predicted at a week-14 evaluation to be non-progressors, i.e., to be complete responders, partial responders, or will be characterized by stable disease. In contrast, those patients having expansion of NK cell counts to less than about 200 cells/μl at 10 weeks post-initiation of therapy are predicted to be progressors, i.e., to have relapse or progressive disease, at the week-14 evaluation. Thus monitoring of NK cell expansion in patients undergoing combination therapy with rituximab and IL-2 can serve as an important diagnostic tool for a patient's prognosis with this therapy.

Thus, the present invention provides a method for treating lymphoma, more particularly non-Hodgkin's B-cell lymphoma in a human subject, comprising administering to the subject at least one therapeutically effective dose of an anti-CD20 antibody and providing a means for maintaining natural-killer (NK) cell count in this subject at or above an acceptable threshold level. This acceptable threshold level is an NK cell count of about 150, preferably an NK cell count of about 175. In some embodiments, the methods effectively maintain an NK cell count in the subject of about 200 or above. The means by which NK cell count is maintained includes any protocol by which IL-2 is administered to the subject such that at least one therapeutically effective dose of IL-2 in an amount that results in an initial IL-2 exposure within a range from about 22 IU*hour/ml serum to about 653 IU*hour/ml serum is administered to the subject, wherein said IL-2 exposure is measured as the area under the serum concentration-time curve (AUC) as determined by human pharmacokinetic (PK) data.

One means for maintaining NK cell count above the acceptable threshold level comprises administering IL-2 according to the constant IL-2 dosing regimen disclosed herein. Thus, the subject is administered at least one therapeutically effective dose of IL-2 in an amount necessary to achieve the same initial IL-2 exposure as a dose of a reference IL-2 standard (i.e., Proleukin® IL-2) in a range from about 666.67 μg to about 1200 μg as determined by the area under the serum concentration-time curve from human PK data. In such an embodiment, the subject is also administered a therapeutically effective dose of anti-CD20 antibody in the range from about 125 mg/m² to about 500 mg/m², which is administered according to a weekly dosing schedule as noted herein above. Where the means for maintaining NK cell count above the acceptable threshold level comprises a constant IL-2 dosing regimen disclosed herein, the therapeutically effective dose of IL-2 can be administered according to a two-times-a-week or three-times-a-week dosing schedule, such that a total weekly dose of IL-2 in an amount equivalent to a total weekly dose of the reference IL-2 standard in a range from 2000 μg to 3600 μg, for example, in a range from 2800 μg to 3600 μg, as determined by the area under the serum concentration-time curve from human PK data is administered to the subject. The duration of dosing of the anti-CD20 antibody can be about 4 weeks to about 8 weeks, and the duration of the constant IL-2 dosing regimen can be about 4 weeks to about 10 weeks, as noted herein above. Further guidance as to particular dosing regimens for the anti-CD20 antibody in combination with constant IL-2 dosing are provided herein above.

Another means for maintaining the NK cell count above the acceptable threshold level comprises administering a two-level dosing regimen of IL-2, where the two-level dosing regimen of IL-2 comprises a first time period, wherein a higher total weekly dose of IL-2 is administered to said subject, followed by a second time period, wherein a lower total weekly dose of IL-2 is administered to said subject, as disclosed herein above. In this embodiment, the higher total weekly dose of IL-2 is in an amount equivalent to a total weekly dose of the reference IL-2 standard in a range from 2000 μg to 3600 μg as determined by the area under the serum concentration-time curve from human pharmacokinetic (PK) data, and the lower total weekly dose of IL-2 is in an amount equivalent to a total weekly dose of a reference IL-2 standard in a range from 1200 μg to about 2600 μg as determined by the area under the serum concentration-time curve from human PK data. As previously noted above, the lower total weekly dose of IL-2 is lower than the higher total weekly dose of IL-2. In such embodiments, the therapeutically effective dose of anti-CD20 antibody is in the range from about 125 mg/m² to about 500 mg/m².

Where the means for maintaining NK cell count above the acceptable threshold level is a two-dose regimen of IL-2, a first dose of IL-2 can be administered to the subject prior to administering a first dose of anti-CD20 antibody, for example, about 1 week to about 30 days prior to administering the first dose of anti-CD20 antibody. Alternatively, a first dose of IL-2 can be administered to the subject concurrently with (i.e., on the same day, either simultaneously or sequentially, in either order) a first dose of anti-CD20 antibody. In yet another embodiment, a first dose of IL-2 is administered to the subject after a first dose of anti-CD20 antibody is administered to the subject, for example, within 10 days, preferably within 7 days, of administering the antibody to the subject. As noted herein above, the higher total weekly dose of IL-2 can be administered as a single dose or can be partitioned into a first series of equivalent doses that are administered according to a two-, three-, four-, five-, six- or seven-times-a-week dosing schedule, and the lower total weekly dose of IL-2 can be administered as a single dose or can be partitioned into a second series of equivalent doses that are administered according to a two-, three-, four-, five-, six- or seven-times-a-week dosing schedule. The duration of dosing of the anti-CD20 antibody can be about 4 weeks to about 8 weeks, and the duration of the two-level IL-2 dosing regimen can be about 4 weeks to about 16 weeks, as noted above. This means of maintaining NK cell count may further comprise giving the subject a drug holiday between the first period of the two-level IL-2 dosing regimen (i.e., where higher total weekly doses of IL-2 are administered) and the second period of the two-level IL-2 dosing regimen (i.e., where lower total weekly doses of IL-2 are administered), as described elsewhere herein.

Where necessary, the subject can be administered multiple maintenance cycles of concurrent therapy with anti-CD20 antibody and the two-level IL-2 dosing regimen to maintain NK cell count above an acceptable threshold level, i.e., about 150. As noted above, each such maintenance cycle would comprise weekly administration of the anti-CD20 antibody in combination with a completed two-level IL-2 dosing regimen (i.e., the subject completes both the first time period of higher total weekly dosing and the second time period of lower total weekly dosing, where the completed two-level IL-2 dosing regimen can further comprise a drug holiday). Further guidance as to particular dosing regimens for the anti-CD20 antibody in combination with a two-level IL-2 dosing regimen are provided herein above.

The following examples are offered by way of illustration and not by way of limitation.

### EXPERIMENTAL

### Example 1

### Phase I Study of Weekly Rituximab Therapy in Combination with Constant Total Weekly Dose of Proleukin® IL-2 in Patients with Non-Hodgkin's Lymphoma

The IL-2 formulation used in this study is manufactured by Chiron Corporation of Emeryville, Calif., under the tradename Proleukin® IL-2. The IL-2 in this formulation is a recombinantly produced, unglycosylated human IL-2 mutein, called aldesleukin, which differs from the native human IL-2 amino acid sequence in having the initial alanine residue eliminated and the cysteine residue at position 125 replaced by a serine residue (referred to as des-alanyl-1, serine-125 human interleukin-2). This IL-2 mutein is expressed in E. coli, and subsequently purified by diafiltration and cation exchange chromatography as described in U.S. Pat. No. 4,931,543. The IL-2 formulation marketed as Proleukin® IL-2 is supplied as a sterile, white to off-white preservative-free lyophilized powder in vials containing 1.3 mg of protein (22 MIU).

The anti-CD20 antibody used in this and the following examples is Rituxan® (rituximab; IDEC-C2B8; IDEC Pharmaceuticals Corp., San Diego, Calif.). It is administered per its package insert dose (375 mg/m² infused over 6 hours).

The primary objective of this study was to determine a maximum tolerated weekly dose (MTD) of Proleukin® IL-2 when administered subcutaneously as three equivalent doses concomitantly with a weekly intravenous (IV) infusion of a fixed dose (375 mg/m²) of Rituxan® (rituximab) for the treatment of CD20+ B-cell non-Hodgkin's lymphoma stage III or IV. The secondary objectives were to explore the effect of IL-2 concomitantly with rituximab on the degree of expansion of natural killer (NK) cells, NK cell function as measured by antibody dependent cytotoxicity (ADCC), anti-tumor responses, the duration of anti-tumor responses, and the pharmacokinetics of IL-2 and rituximab.

Study Design

This was an open-label study of escalating doses of IL-2 in combination with 375 mg/m² rituximab infused once weekly for a total of 4 doses. Multiple study centers are anticipated. The total weekly doses of IL-2 were 13.5 million international units (i.e., thrice-weekly IL-2 dose of 4.5 MIU), 30.0 MIU (i.e., thrice-weekly IL-2 dose of 10.0 MIU), 42.0 MIU (i.e., thrice-weekly IL-2 dose of 14.0 MIU), and 54.0 MIU (i.e., thrice-weekly IL-2 dose of 18.0 MIU). These correspond to relative total weekly doses of about 7.9 MIU/m², about 17.6 MIU/m², about 24.7 MIU/m², and about 31.8 MIU/m². The total weekly dose of Proleukin® IL-2 (referred to hereafter in this example as IL-2) was partitioned into three equivalent doses that were administered three times weekly (tiw) by subcutaneous injection concomitantly with weekly IV infusions of rituximab. Interleukin-2 treatments began 1 week after the first IV infusion of rituximab and continued up through the end of week 5. Patients remained on a fixed dose of IL-2 throughout this period.

Treatments Administered and Dose Escalation Methodology

Patients began treatment with 375 mg/M² of rituximab by IV infusion on day 1 and then weekly for 3 additional weeks. Thrice-weekly subcutaneous injections of IL-2 started on day 8 at the assigned dose and continued a total of 4 weeks. Thrice-weekly treatment of IL-2 was defined as administration of IL-2 three times per week with at least 48 hours between injections. First injections of IL-2 were given within 30 minutes after the start of the rituximab infusion. The following two IL-2 injections were administered in 48-hour intervals. Weeks 2 through 4 of IL-2 subcutaneous injections began concomitantly with rituximab infusion. Week 5 was IL-2 subcutaneous injections only. The thrice-weekly IL-2 dose levels studied were 4.5 million international units (MIU), 10.0 MIU, 14.0 MIU and 18.0 MIU, which correspond to total weekly doses of 13.5 MIU, 30.0 MIU, 42.0 MIU, and 54.0 MIU.

A dose limiting toxicity (DLT) is defined as a treatment-related adverse reaction with toxicity grading of grade 3 or grade 4 by National Cancer Institute (NCI) criteria (i.e., NCI Common Toxicity Criteria), with the exception of the hematologic and fever toxicities, which require a toxicity grading of grade 4 to be considered a DLT. Some specific criteria that may be encountered during the course of the study include grade 3 toxicities (for example, white blood cell count (a value of 1.0-<2.0×10³/μl), platelets (a value of 25-49×10³/μl), hemoglobin (a value of 6.5-<8.0 g/dl), infection (severe, not life threatening), vomiting (6-10 episodes in 24 hours in the presence of sufficient anti-emetic therapy), pulmonary (dyspnea at normal levels of exertion), hypotension (requiring therapy and hospitalization; resolves within 48 hours of stopping study medications), neurosensory (severe objective sensory loss or paresthesias that interfere with function), neuromoter (objective weakness with impairment of function), fever (oral greater than 39.6-40.4° C.), fatigue (normal activity decreased greater than 50% inability to work), weight gain on study (at least 20.0%), local reactions (induration greater than 10 cm²; ulceration or necrosis that is severe or prolonged), etc.), and grade 2 toxicities (for example, cardiac dysrhythmia (recurrent or persistent but not requiring therapy), cardiac function (decline of resting ejection fraction by more than 20%), cardiac ischemia (asymptomatic ST-T wave changes), and pericardium (pericarditis by clinical criteria). Except for what is listed herein, any grade 3 toxicity is considered dose limiting. Specific examples of adverse reactions that must be a grade 4 to be considered a DLT are absolute neutrophil count (ANC)<5×10²/μl); total white blood cell count (WBC)<1×10³/μl; hemoglobin (Hgb)<6.5 g/dl; platelets<25×10³/μl; and fever (oral) greater than 40.5° C. or 105° F.

Cohorts of 3 patients were enrolled at each IL-2 dose level. Adverse events of dose-limiting toxicity (DLT) were monitored in patients through the end of week 5. If the current dose level of IL-2 was tolerated by all 3 patients through the end of week 5 without any adverse events of DLT, another cohort of three patients was enrolled at the next higher dose level of IL-2. Subjects at this next dose level could be enrolled based on the DLT data at week 3 of IL-2 (week 4 of the study) and receive rituximab, however IL-2 was not administered until all patients within the cohort had completed the 5-week regimen. If one of the 3 patients experienced an adverse event of DLT at any time during the 5-week regimen, 3 additional patients were enrolled at this dose level. The dose of IL-2 was not increased to the next dose level unless the 3 additional patients completed week 5 without experiencing an adverse event of DLT. Subjects at this next dose level could be enrolled and receive rituximab, however IL-2 was not administered until all patients within the cohort had completed the 5-week regimen. The outpatient MTD of IL-2 was considered to be the dose level immediately below the lowest dose level at which adverse events of DLT were observed in 2 or more patients.

Selection of Study Population

The primary inclusion criteria for patients enrolled in this study were: documentation of CD20+ B cell non-Hodgkin's lymphoma stage III or IV; patients should be relapsed or refractory to first-line treatments; Karnofsky Performance Score of ≧70%; and not less than 18 years old. Patients were excluded from the study for the following reasons: prior treatment with IL-2; prior treatment with rituximab for any indication within 3 months of study treatment; and current or prior medical history inconsistent with use of rituximab or IL-2.

Measurements, Safety, and Efficacy

The primary objective of the study was to determine the MTD of thrice-weekly IL-2 when administered concomitantly with weekly doses of 375 mg/m² rituximab. For the purpose of estimating MTD, patients could not miss more than one dose of IL-2 consecutively, nor miss more than 30% or more of the prescribed IL-2 dose, and had to receive all 4 doses of rituximab in order to be included in the analysis (unless patients experience DLTs while on IL-2).

It was of importance to the success of the proposed combination therapy to establish that NK cell number and function and T cell number were enhanced during the course of the study. These cell types may be expanded after treatment with IL-2, which may be essential for rituximab anti-tumor activity. Therefore, measurements of NK cell number and function and T cell subset numbers were performed. In this manner, lymphocyte subsets (the percent and absolute number of lymphocytes expressing CD3 CD4, CD8, CD16+56, and CD19, and the percent of lymphocytes expressing CD20) and NK cell ADCC function were measured at weekly intervals throughout the study prior to rituximab infusion using standard protocols. NK cell expansion appears to be a critical requirement for enhancement of rituximab activity, and the extent of NK cell expansion is a component in subsequent dosing decisions in future studies. Other variables observed in this study included tumor response and duration and the pharmacokinetics of IL-2.

Efficacy was assessed in all patients as a secondary variable. An evaluable patient was defined as: subjects must have received 4 weeks of rituximab therapy and 70% of the prescribed Proleukin® IL-2 dose and schedule. The response was evaluated as follows. Tumor measurements were based upon measurements of perpendicular diameters, using the longest diameter and its greatest perpendicular. Grading of tumor response is based upon the report of the International Workshop to Standardize Response Criteria for Non-Hodgkin's Lymphomas (see, Cheson et al. (1999) J. Clin. Oncol. 17:1244-1253) and protocol-defined criteria as follows:

Complete response (CR)—Defined as absence of clinically detectable disease with normalization of any previously abnormal radiographic studies, bone marrow and cerebrospinal fluid (CSF). Response must persist for at least one month. Patients with bone marrow positive for lymphoma prior to chemotherapy must have a repeat biopsy, which is confirmed after a month, negative for lymphoma.

Partial response (PR)—Defined as at least 50% decrease in all measurable tumor burden in the absence of new lesions and persisting for at least one month (applicable to measurable tumors only).

Patients were also assessed for effects of Proleukin® IL-2 and rituximab therapy on the following:

Response duration—Defined as the time from first documented response until progressive disease.

Time to progression—Defined as the time from study entry to progressive disease, relapse or death.

Stable disease (SD)—Defined as a less than 50% reduction in tumor burden in the absence of progressive disease.

Progressive disease (PD)—Defined as representing 25% or greater increase in tumor burden or the appearance of a new site of the disease.

Relapse (R)—Defined as the appearance of tumor following documentation of a complete response.

Evaluation of efficacy by tumor response was a secondary objective. Efficacy was also measured by the degree of expansion of NK cells; NK cell function as measured by ADCC; anti-tumor responses; and the duration of anti-tumor responses.

In total, 15 patients have been enrolled in the phase I clinical trial described above. The thrice-weekly dosing regimen was generally well tolerated through the 14 MIU dose level. At the 18 MIU dose, 3 patients completed therapy at the full dose. A fourth patient developed dose-limiting toxicity (hypersensitivity), and further treatment was stopped.

Thirteen patients have completed therapy and have been evaluated at week 14 for best tumor response. Overall, 7 patients responded, 4 with complete response (CR), and 3 with partial response (PR); 4 patients had stable disease (SD); and 2 had progressive disease (PD). All 7 of the responders received one of the three highest doses on the thrice-weekly schedule. Two CRs occurred in patients with intermediate- or high-grade lymphoma, and 2 CR occurred in patients with low-grade lymphoma. The 3 PRs were seen in patients with intermediate- or high-grade, including one with mantle-cell lymphoma.

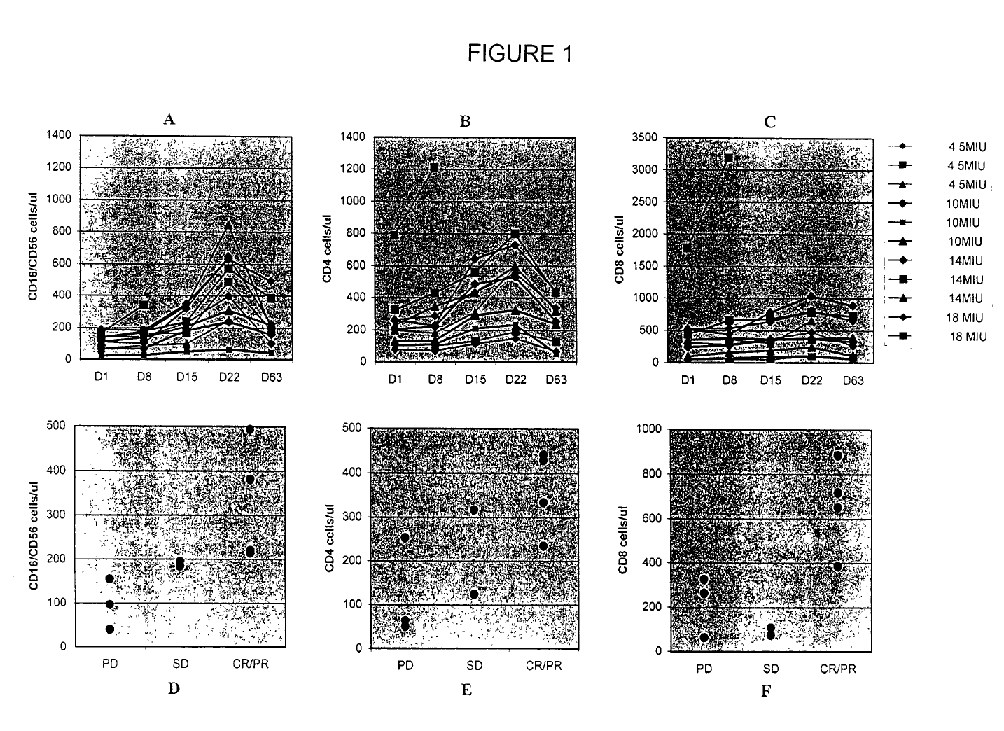

FIG. 1 shows the time course for natural killer (NK) cell count (CD16/CD56 cells) (Panel A), CD4 cell count (Panel B), and CD8 cell count (Panel C) in 11 patients undergoing concurrent therapy with weekly rituximab therapy (375 mg/m2) and thrice-weekly doses of Proleukin® IL-2 (recombinant human IL-2 mutein) for treatment of non-Hodgkin's lymphoma. NK cell count was determined by flow cytometry. Rituximab was administered by infusion over up to 6 hours on day 1 (D1), day 8 (D8), day 15 (D15), and day 22 (D22). Proleukin® IL-2 was administered subcutaneously three times per week for 4 weeks beginning on day 8. The doses of Proleukin® IL-2 were 4.5 MIU (3 patients), 10 MIU (3 patients), 14 MIU (3 patients), and 18 MIU (2 patients). The corresponding cell counts at week 10 for the 9 patients with available data that have completed the entire dosing regimen versus their clinical response to therapy at week 14 are shown in Panel D (NK cell count), Panel E (CD4 cell count) and Panel F (CD8 cell count). PD=progressive disease; SD=stable disease; CR/PR=complete response or partial response.

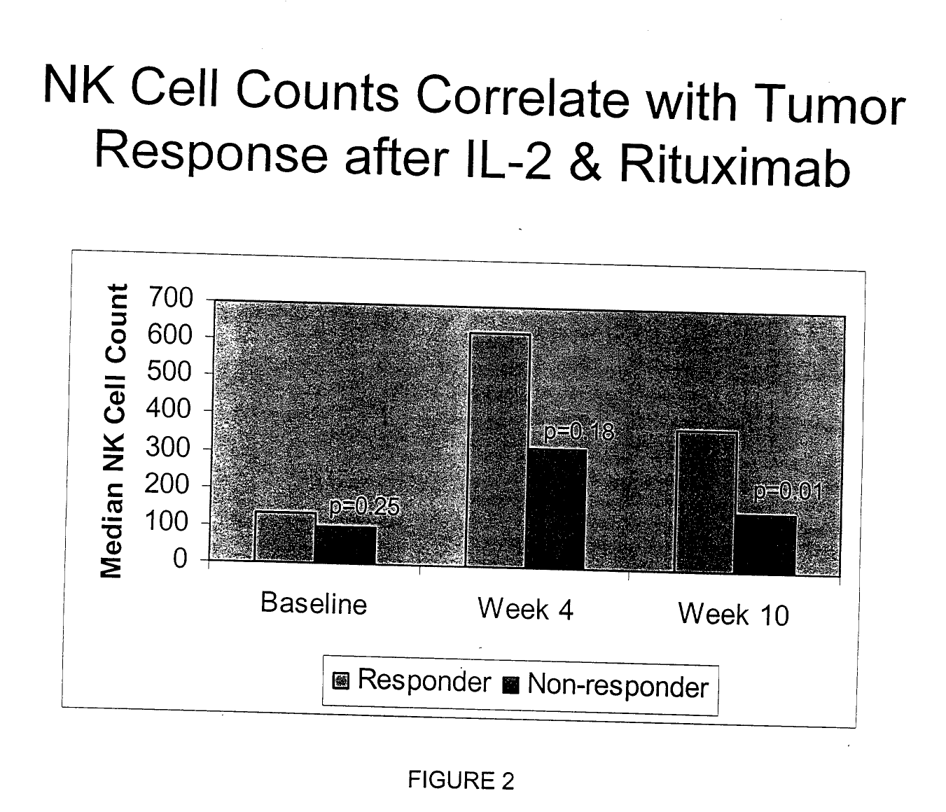

Natural killer (NK) CD56+CD 16+ cell numbers increased with thrice-weekly IL-2 administration in all except one patient, who had disease progression (FIG. 1, Panel A). At the beginning of week 4, 4 out of 5 responders had absolute NK cell counts above 572 cells/μl, while the 4 patients with progressive disease had NK cell counts less than 394 cells/μl (individual patient data not shown; for median cell count, see FIG. 2). For the 10 patients for whom NK cell values are currently available at week 10, the degree of NK CD56+CD16+ cell expansion during IL-2 therapy on the thrice-weekly schedule was higher in responders (complete responders and partial responders) than in non-responders (stable disease and progressive disease patients), with a median of 382 cells/μl (range 215-494 cells/μl) compared to 155 cells/μl (range 40-195 cells/μl) (FIG. 1, Panel D; see also FIG. 2). The median NK cell count at week 10 showed a statistically significant difference between these groups (p=0.01; FIG. 2).

Of the three populations of cell counts collected, NK cell count appears to be an important predictor to response with rituximab when combined with IL-2. When the number of NK cells is plotted against the tumor response at week 14 to this thrice-weekly dosing of IL-2 with weekly dosing of rituximab, it can be seen that those patients having stable disease or a complete response or partial response have at least 185 cells/μl at 10 weeks post-initiation of rituximab therapy (FIG. 1, Panel D; FIG. 2).

In addition, fresh ex vivo NK-mediated natural cytolytic killing against the NK-sensitive, LAK-resistant K562 cell line, and LAK and ADCC-mediated function against the NK-resistant, LAK-sensitive Daudi B-cell line in the presence and absence of optimal concentrations of rituximab in a standard 4-hr ⁵¹Cr-release cytotoxicity assay at effector:target ratios of 50:1-1.56:1 have been evaluated for responders and non-responders in both IL-2 dosing protocols. For a description of this cytotoxicity assay, see, for example, Vlasveld et al. (1995) Cancer Immunol. Immunother. 40(1):37-47. For this study, PBMC were isolated by Ficoll Hypague density centrifugation from the blood of patients enrolled in the program. PBMC were isolated prior to rituximab treatment (day 1; d1), pre-IL-2 treatment (day 8; d8), after one week of IL-2 treatment (day 15; d15), after two weeks of IL-2 treatment (day 22; d22), and at subsequent time points throughout the study. PBMC were tested at an effector to target ratio (E:T) range of 50:1-1.56:1 against a panel of ⁵¹Cr-labeled target cells comprised of K562 cells, Daudi cells, and Daudi cells in the presence of optimal concentrations of rituximab (2 μg/ml) to evaluate NK, LAK, and ADCC activity, respectively. ⁵¹Cr release was measured after a 4-hour incubation period.

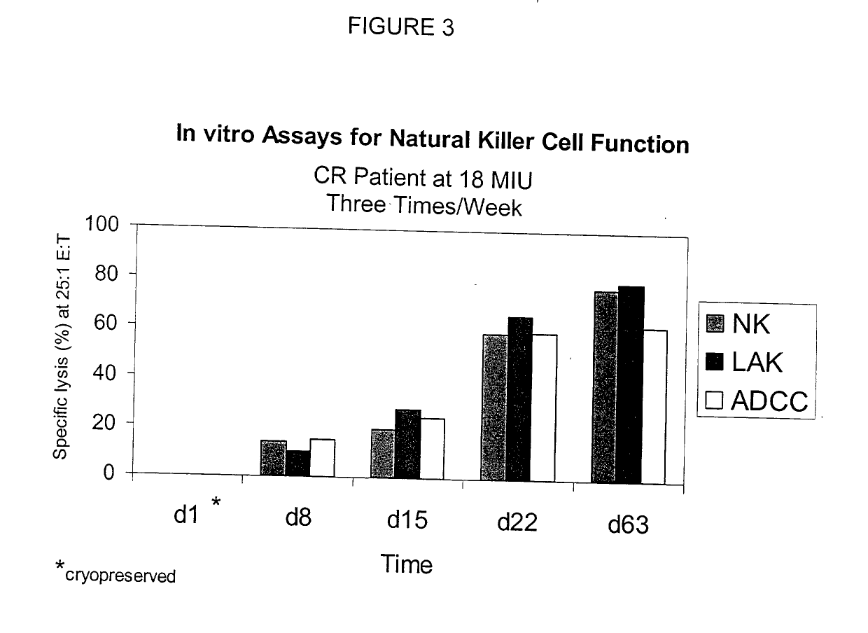

Responders in this protocol demonstrated sustained NK-mediated activity, as noted by NK natural cytotoxicity, LAK, and ADCC-mediated killing, that was increased progressively and was maintained at week 10, despite the fact that IL-2 dosing was completed by the end of week 5 (FIG. 3). This trend was also observed for patients with stable disease, though to a lesser extent than observed for responders (data not shown). In contrast, those who developed progressive disease exhibited lower transient levels of NK-mediated killing activity, which declined rapidly following cessation of IL-2 treatment (data not shown). Collectively, the NK cell count data and NK function data suggest that IL-2-mediated NK cell expansion and function are critical interdependent determinants of clinical response outcome to concurrent therapy with IL-2 and rituximab.

Thus, when undergoing concurrent therapy with IL-2 and rituximab as outlined herein with thrice-weekly dosing for 4 weeks, those patients having expansion of NK cell counts to greater than about 170 cells/μl at 10 weeks post-initiation of therapy are predicted to be complete responders, partial responders, or are characterized by stable disease. In contrast, those patients having expansion of NK cell counts to less than about 170 cells/μl at 10 weeks post-initiation of therapy are predicted to have relapse or progressive disease. Thus, monitoring of NK cell expansion in patients undergoing concurrent therapy with rituximab and IL-2 can serve as an important diagnostic tool for a patient's prognosis with this therapy.

### Example 2

### Phase II/III Clinical Trial with Weekly Rituximab Therapy for 4 Weeks in Combination with 8-week Two-Level IL-2 Dosing Regimen of Proleukin® IL-2 in Patients with Non-Hodgkin's Lymphoma who have Previously Failed Rituximab

A phase II/III clinical trial is carried out to evaluate safety and efficacy of a 4-week rituximab therapy (i.e., weeks 1-4) in combination with an 8-week two-level IL-2 dosing regimen of Proleukin IL-2 (weeks 2-9) for the treatment of CD20+B-cell non-Hodgkin's lymphoma in patients who previously failed to respond to rituximab or relapsed within 6 months of treatment. The secondary objectives are to further document the effect of IL-2 concomitantly with rituximab on the degree of expansion of natural killer (NK) cells, NK cell function as measured by antibody dependent cytotoxicity (ADCC), anti-tumor responses, the duration of anti-tumor responses, and the pharmacokinetics of IL-2 and rituximab.

Study Design

This is an open-label study employing two doses of IL-2 in combination with 375 mg/m² rituximab infused once weekly (6-hour infusion). Patients are administered a weekly IV infusion of a fixed dose (375 mg/m²) of Rituxan® (rituximab) beginning on day 1 of each week for a period of 4 weeks (i.e., a total of 4 doses). Thus rituximab is administered on days 1, 8, 15, 22. Patients begin concomitant administration of Proleukin® IL-2 (hereinafter referred to as reference IL-2 standard) by subcutaneous injection on day 1 of the second week (i.e., day 8 of the treatment period). The total weekly dose of IL-2 is partitioned into three equivalent doses that are administered according to a three-times-per-week dosing schedule, with a minimum of 48 hours between administrations, for a period of 8 weeks (i.e., total of 24 doses during weeks 2-9 of the treatment period). During weeks 2-5, the total weekly IL-2 dose to be administered as three equivalent doses is 42.0 MIU (i.e., each equivalent dose is 14.0 MIU). After 4 weeks of IL-2 administration, the total weekly IL-2 dose is lowered to 30.0 MIU. Thus, during weeks 6-9, a total weekly IL-2 dose of 30.0 MIU is partitioned into three equivalent doses (i.e., each 10.0 MIU) that are administered according to the three-times-per-week dosing schedule. Patients are monitored for efficacy and safety of this treatment regimen throughout the 9-week treatment period, with follow-up determinations occurring through week 16 (i.e., for 7 weeks beyond the last week of IL-2 administration).

Selection of Study Population

Patients are eligible if they have CD20+, B-cell, non-Hodgkin's lymphoma of low-grade or follicular histology with measurable relapsed or unresponsive disease after prior therapy. In addition, they must have previously received a course of single-agent rituximab and showed no tumor response, or had a response lasting <6 months. The previously administered rituximab must have included at least 75% of the standard 4-week regimen (4×375 mg/m²). A record of the previous rituximab treatment and response must be available as a source document at the site. Other primary inclusion and exclusion criteria are similar to those noted for the phase I clinical trial described in Example 2 above.

Measurements Safety, and Efficacy

The primary objective of the study is to determine the safety and efficacy of thrice-weekly IL-2 when administered for 8 consecutive weeks concomitantly with weekly doses of 375 mg/m² rituximab. Patients must not miss more than one dose of IL-2 consecutively, nor miss more than 30% or more of the prescribed IL-2 dose, and receive all 4 doses of rituximab in order to be included in the primary efficacy analysis.

Efficacy is assessed by tumor response and duration of tumor response using the procedures and criteria noted for the phase I clinical trial described in Example 1. Tumor response is correlated with increases in NK cells determined by flow cytometry. Other variables observed in this study in a subset of patients are NK cell function and the pharmacokinetics of IL-2, as noted for the phase I clinical trial described in Example 1.

### Example 3

### Weekly Rituximab Therapy for 8 Weeks in Combination with an 8-Week Two-Level IL-2 Dosing Regimen of Proleukin® IL-2 in Patients with Aggressive Non-Hodgkin's Lymphoma

A dosing schedule similar to that outlined in Example 2 is carried out with subjects having aggressive non-Hodgkin's Lymphoma (stage III or IV, i.e., intermediate- to high-grade), with the exception of extending the weekly rituximab therapy out to 8 weeks. In this manner, subjects are given an 8-week rituximab therapy (i.e., weeks 1-8) in combination with an 8-week two-level IL-2 dosing regimen of Proleukin® IL-2 (weeks 2-9). The secondary objectives are to further document the effect of IL-2 concomitantly with rituximab on the degree of expansion of natural killer (NK) cells, NK cell function as measured by antibody dependent cytotoxicity (ADCC), tumor response, and the duration of tumor response.

Eligible subjects are administered a weekly IV infusion of a fixed dose (375 mg/m²) of Rituxan® (rituximab) beginning on day 1 of each week for a period of 8 weeks (i.e., a total of 8 doses). Thus rituximab is administered on days 1, 8, 15, 22, 29, 36, 43, and 50. Subjects begin concommitant administration of Proleukin® IL-2 (hereinafter referred to as reference IL-2 standard) by subcutaneous injection on day 1 of the second week (i.e., day 8 of the treatment period). The total weekly doses of IL-2 are partitioned into three equivalent doses that are administered according to a three-times-per-week dosing schedule, with a minimum of 48 hours between administrations, for a period of 8 weeks (i.e., total of 24 doses during weeks 2-9 of the treatment period). During weeks 2-5, the total weekly IL-2 dose to be administered as three equivalent doses is 42.0 MIU (i.e., each equivalent dose is 14.0 MIU). After 4 weeks of IL-2 administration, the total weekly IL-2 dose is lowered to 30.0 MIU. Thus, during weeks 6-9, a total weekly IL-2 dose of 30.0 MIU is partitioned into three equivalent doses (i.e., each 10.0 MIU) that are administered according to the three-times-per-week dosing schedule. Subjects are monitored for efficacy and safety of this treatment regimen throughout the 9-week treatment period, with follow-up determinations occurring through week 16 (i.e., for 7 weeks beyond the last week of IL-2 administration).

### Example 4

### Phase I Clinical Trial with Weekly Rituximab Therapy for 4 Weeks in Combination with 4-Week Constant Total Weekly Dose of Monomeric IL-2 in Patients with Non-Hodgkin's Lymphoma

A phase I clinical trial is carried out to examine the use of a monomeric formulation of IL-2, L2-7001, for the treatment of CD20+ B-cell non-Hodgkin's lymphoma. The particular monomeric IL-2 formulation to be used is L2-7001. This liquid formulation comprises the same human IL-2 mutein (aldesleukin) as Proleukin® IL-2 with the exception of the final purification steps prior to its formulation. As noted in Example 1 above, this IL-2 mutein is expressed from E. coli. The initial purification steps to obtain aldesleukin are similar for the two formulations. See U.S. Pat. No. 4,931,543. In both cases, the recombinantly produced IL-2 mutein occurs as refractile bodies within the host cells. Following cell disruption, the refractile bodies are isolated and initially purified using size exclusion chromatography and RP-HPLC. The remaining purification steps for the IL-2 mutein used in L2-7001 are as follows. The resulting protein precipitate is solubilized by guanidine hydrochloride, then processed by diafiltration, ion exchange chromatography, and subsequent diafiltration to obtain the final purified IL-2 mutein for use in making the L2-7001 formulation. In contrast, when this IL-2 mutein is used in Proleukin® IL-2, the protein precipitate resulting from the initial purification steps is solubilized by 1% SDS, then processed by size exclusion chromatography and dialfiltration. The purified IL-2 mutein is then formulated into L2-7001 according to the method disclosed in the copending application entitled “Stabilized Liquid Polypeptide-Containing Pharmaceutical Compositions,” filed Oct. 3, 2000, and assigned U.S. application Ser. No. 09/677,643.

This is an MTD dose-finding study similar to that described for Proleukin® IL-2. In this study, a constant total weekly dose of L2-7001 is administered over a 4-week period in combination with 4 weekly doses of rituximab at its recommended dose (i.e., 375 mg/m²). The total weekly IL-2 doses are partitioned into three equivalent doses that are administered according to a three-times-a-week dosing schedule, with a minimum of 48 hours between administrations. The dose escalation methodology is similar to that described before. The initial escalating total weekly doses of L2-7001 are 540 μg, 810 μg, 1080 μg, and 1500 μg as determined from AUC data for pharmacokinetics of L2-7001. See Table 4 in Example 8 below. Study design and data collected are similar to those described in Example 1 above. Safety and efficacy are evaluated as noted in the clinical trials described above.

### Example 5

### Weekly Rituximab Therapy for 4 Weeks in Combination with 8-week, Two-Dose Regimen of L2-7001 in Patients with Non-Hodgkin's Lymphoma who have Previously Failed Rituximab

As an alternative to the dosing regimen outlined in Example 2, eligible subjects are administered the monomeric IL-2 formulation L2-7001, instead of Proleukin® IL-2. In this manner, subjects are administered a weekly IV infusion of a fixed dose (375 mg/m²) of Rituxan® (rituximab) beginning on day 1 of each week for a period of 4 weeks (i.e., a total of 4 doses). Thus rituximab is administered on days 1, 8, 15, 22. Subjects begin concomitant administration of L2-7001 (hereinafter referred to as IL-2 in this example) by subcutaneous injection on day 1 of the second week (i.e., day 8 of the treatment period). The total weekly dose of IL-2 is partitioned into three equivalent doses that are administered according to a three-times-per-week dosing schedule, with a minimum of 48 hours between administrations, for a period of 8 weeks (i.e., total of 24 doses during weeks 2-9 of the treatment period). During weeks 2-5, the total weekly IL-2 dose to be administered as three equivalent doses is 810 μg (i.e., each equivalent dose is 270 μg). After 4 weeks of IL-2 administration, the total weekly IL-2 dose is lowered to 540 μg. Thus, during weeks 6-9, a total weekly IL-2 dose of 540 μg is partitioned into three equivalent doses (i.e., each 180 μg) that are administered according to the three-times-per-week dosing schedule. Subjects are monitored for efficacy and safety of this treatment regimen throughout the 9-week treatment period, with follow-up determinations occurring through week 16 (i.e., for 7 weeks beyond the last week of IL-2 administration).

### Example 6

### Phase II/III Clinical Trial with Weekly Rituximab Therapy for 8 Weeks in Combination with 8-week Two-Dose Regimen of L2-7001 in Patients with Aggressive Non-Hodgkin's Lymphoma

As an alternative to the dosing regimen outlined in Example 3, eligible subjects are administered the monomeric IL-2 formulation L2-7001, instead of Proleukin® IL-2. a weekly IV infusion of a fixed dose (375 mg/m²) of Rituxan® (rituximab) beginning on day 1 of each week for a period of 8 weeks (i.e., a total of 8 doses). Thus rituximab is administered on days 1, 8, 15, 22, 29, 36, 43, and 50. Patients begin concommitant administration of L2-7001 (hereinafter referred to as IL-2) by subcutaneous injection on day 1 of the second week (i.e., day 8 of the treatment period). The total weekly doses of IL-2 are partitioned into three equivalent doses that are administered according to a three-times-per-week dosing schedule, with a minimum of 48 hours between administrations, for a period of 8 weeks (i.e., total of 24 doses during weeks 2-9 of the treatment period). During weeks 2-5, the total weekly IL-2 dose to be administered as three equivalent doses is 810 μg (i.e., each equivalent dose is 270 μg). After 4 weeks of IL-2 administration, the total weekly IL-2 dose is lowered to 540 μg. Thus, during weeks 6-9, a total weekly IL-2 dose of 540 μg is partitioned into three equivalent doses (i.e., each 180 μg) that are administered according to the three-times-per-week dosing schedule. Subjects are monitored for efficacy and safety of this treatment regimen throughout the 9-week treatment period, with follow-up determinations occurring through week 16 (i.e., for 7 weeks beyond the last week of IL-2 administration).

### Example 7

### Calculating Equivalent Doses for Proleukin® IL-2 in Different Units of Measure

The foregoing doses of Proleukin® IL-2 used in the phase I and phase II clinical trials represent absolute doses in MIU. One can readily determine the corresponding relative dose in MIU/m² as the average person is approximately 1.7 m². Similarly, one can determine the corresponding absolute dose in microgram units given that Proleukin® IL-2 has a biological activity of about 15 MIU per mg of this IL-2 product. Table 2 provides equivalent total weekly doses for Proleukin® IL-2 in different units of measure.

TABLE 2
Equivalent total weekly doses for
Proleukin ® IL-2 in different units of measure.

| MIU/m² | MIU | Micrograms (μg) |
|---|---|---|
| 10.6 | 18.0 | 1200.00 |
| 14.7 | 25.0 | 1666.67 |
| 17.6 | 30.0 | 2000.00 |
| 20.6 | 35.0 | 2333.33 |
| 22.9 | 39.0 | 2600.00 |
| 24.7 | 42.0 | 2800.00 |
| 29.4 | 50.0 | 3333.33 |
| 31.8 | 54.0 | 3600.00 |

### Example 8

### Calculation IL-2 Serum Concentration-Time Curves for Pharmaceutical Formulations of IL-2

The area under the serum concentration-time curve (AUC) of Proleukin® IL-2 administered subcutaneously (SC) at 4.5 million international units (MIU) (equivalent to approximately 300 μg protein) was determined using data from an unpublished HIVstudy. Serum concentration time profiles were measured in 8 IL-2 naïve, HIV patients following an initial exposure to IL-2 dosing in this study. For each patient, the AUC was calculated using the linear trapezoidal rule up to the last measurable concentrations and extrapolated to 24 hours (Winnonlin software version 3.1, Pharsight Corporation, California). The average AUC₀₋₂₄, SD, and the lower and upper 95% confidence limits at 4.5 MIU dose are presented in Table 3.

The AUC₀₋₂₄ value of Proleukin® IL-2 administered SC at doses equivalent to 18 MIU (1200 μg) was estimated using data from three different studies where this IL-2 product was administered SC. Two are published studies, one in HIV patients (N=3) (Piscitelli et al. (1996) Pharmacotherapy 16(5):754-759) and one in cancer patients (N=7) (Kirchner et al. (1998) Br. J. Clin. Pharmacol. 46:5-10). The third is an unpublished study in which serum concentration time data were available from 6 cancer patients after SC doses of IL-2. The similarity of the AUC in cancer and HIV patients was previously established (unpublished data). The actual doses administered in these three studies ranged between 18 and 34 MIU. For the two published trials, the AUC up to 24 hours (AUC₀₋₂₄) values were normalized to 18 MIU dose by multiplying the AUC with the quotient of 18 and actual dose in MIU. For example, if the AUC₀₋₂₄ for a 20 MIU dose was calculated to be 400, the normalized AUC₀₋₂₄ would be 400*18/20=360. For the unpublished cancer-patient study, individual AUC values were calculated from the serum concentration time data using the linear trapezoidal rule up to the last measurable concentrations and extrapolated to 24 hours (Winnonlin software version 3.1, Pharsight Corporation, California) then were normalized to 18 MIU dose as noted above. The overall mean and SD for all three studies was calculated as the weighted average of the means and variances, respectively, using equations 1 and 2.

$$\begin{matrix}{\overline{X}}_{P} = \frac{( {n}_{1} {\overline{X}}_{1} + {n}_{2} {\overline{X}}_{2} + {n}_{3} {\overline{X}}_{3} )}{( {n}_{1} + {n}_{2} + {n}_{3} )} . & 1 \\ {SD}_{P} = \sqrt{\frac{( {n}_{1} - 1 ) {s}_{1}^{2} + ( {n}_{2} - 1 ) {s}_{2}^{2} + ( {n}_{3} - 1 ) {s}_{3}^{2}}{( {n}_{1} + {n}_{2} + {n}_{3} - 3 )}} . & 2\end{matrix}$$

Where n₁, n₂, n₃, \overline{X}₁, \overline{X}₂, \overline{X}₃ and s₁², s₂², s₃² are the number of subjects, means, and variances for each of the three studies, respectively. \overline{X}_{P} and SD_{P} are estimates of the overall mean and standard deviation. The overall average AUC, SD, and the lower and upper 95% confidence limits at 18 MIU are also presented in Table 2.

TABLE 3
Average (±SD) AUC₀₋₂₄ obtained after initial exposure to a single dose
administration of Proleukin ® IL-2 administered subcutaneously.

| Proleukin ® IL-2 | | | | |
|---|---|---|---|---|
| Dose | AUC₀₋₂₄ | | | |
| (MIU/μg) | (IU*hr/ml) | SD | LL of 95% CI¹ | UL of 95% CI¹ |
| 4.5/300 | 56 | 15 | 26 | 86 |
| 6.0/400² | 71 | 24 | 117 | |
| 7.5/500 | 86 | 31.5 | 22.5 | 148.5 |
| 18/1200 | 375 | 139 | 97 | 653 |

¹Upper (UL) and lower (LL) limits of the 95% confidence intervals (CI). 95% CI were calculated as the mean ± 2 SD.
²Values for 6.0 MIU are estimated based on actual values for 4.5 MIU and 7.5 MIU.

Similar to Proleukin® IL-2, L2-7001, a liquid formulation of monomeric IL-2, was administered to HIV patients at doses ranging from 50 to 180 μg (unpublished data). The exposures obtained from this study as measured by AUC are shown in Table 4. These exposure values were within the range of the exposure values generated using Proleukin® IL-2 (Table 3).

TABLE 4
Average (±SD) AUC₀₋₂₄ obtained after an initial exposure to a single dose
administration of the monomeric IL-2 formulation L2-7001.

| L2-7001 Dose | AUC₀₋₂₄ | |
|---|---|---|
| (MIU/μg) | (IU*hr/ml) | SD |
| 0.75/50 | 65 | 12 |
| 1.35/90 | 120 | 39 |
| 2.0/135 | 156 | 45 |
| 2.7/180 | 300 | 108 |

The IL-2 exposure data (AUC) was obtained from the published literature where recombinant human native IL-2 was administered SC to 8 cancer patients at doses ranging from 0.1 MU to 3.0 MU. The reported average (% CV) AUCs for the 0.3, 1, and 3 MU dose levels were 120 (38), 177 (36), and 359 (46) U*hr/ml (Gustavson (1998) J. Biol. Response Modifiers 1998:440-449). As indicated in Thompson et al. 1987 Cancer Research 47:4202-4207, the units measured in this study were normalized to BRMP units (Rossio et al. (1986) Lymphokine Research 5 (suppl 1):S13-S18), which was adopted later as international units (IU) by WHO (Gearing and Thorpe (1988) J. Immunological Methods 114:3-9). The AUC values generated under the study conditions also agree well with the established Proleukin® IL-2 exposure.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Those skilled in the art will recognize, or be able to ascertain using no more than routine experimentation, many equivalents to the specific embodiments of the invention described herein. Such equivalents are intended to be encompassed by the following claims.

That which is claimed:

1. A method of treating a non-Hodgkin's B-cell lymphoma in a human subject, said method comprising administering to said subject at least one maintenance cycle of concurrent therapy with an anti-CD20 antibody and interleukin-2 (IL-2), wherein said maintenance cycle comprises administering a therapeutically effective weekly dose of an anti-CD20 antibody in combination with administration of a two-level dosing regimen of IL-2, said two-level dosing regimen of IL-2 comprising a first time period, wherein a higher total weekly dose of IL-2 is administered to said subject, followed by a second time period, wherein a lower total weekly dose of IL-2 is administered to said subject.

2. The method of claim 1, wherein a first dose of IL-2 is administered to said subject prior to administering a first dose of anti-CD20 antibody.

3. The method of claim 2, wherein said first dose of IL-2 is administered up to one month before the first dose of anti-CD20 antibody is administered to said subject.

4. The method of claim 3, wherein said first dose of IL-2 is administered one week before the first dose of anti-CD20 antibody is administered to said subject.

5. The method of claim 1, wherein a first dose of IL-2 is administered to said subject concurrently with a first dose of anti-CD20 antibody.

6. The method of claim 1, wherein a first dose of IL-2 is administered to said subject one week after a first dose of anti-CD20 antibody is administered to said subject.

7. The method of claim 1, wherein said anti-CD20 antibody is dosed weekly for 4 weeks to 8 weeks.

8. The method of claim 7, wherein said therapeutically effective dose of said anti-CD20 antibody is in the range from about 125 mg/m² to about 500 mg/m².

9. The method of claim 1, wherein said two-level dosing regimen of IL-2 has a combined duration of 4 weeks to 16 weeks.

10. The method of claim 9, wherein said first time period of said two-level dosing regimen of IL-2 has a duration of at least 1 week out of said combined duration of 4 weeks to 16 weeks.

11. The method of claim 9, wherein said first time period of said two-level dosing regimen of IL-2 has a duration that is one-half of said combined duration of 4 weeks to 16 weeks.

12. The method of claim 1, wherein said higher total weekly dose of IL-2 is administered as a single dose or is partitioned into a first series of equivalent doses that are administered according to a two-, three-, four-, five-, six- or seven-times-a-week dosing schedule, and wherein said lower total weekly dose of IL-2 is administered as a single dose or is partitioned into a second series of equivalent doses that are administered according to a two-, three-, four-, five-, six- or seven-times-a-week dosing schedule.

13. The method of claim 12, wherein said IL-2 is administered by a route selected from the group consisting of intravenous, intramuscular, and subcutaneous.

14. The method of claim 12, wherein said higher total weekly dose of IL-2 is administered as a single dose.

15. The method of claim 12, wherein said first series of equivalent doses is administered according to a two-times-a-week dosing schedule.

16. The method of claim 12, wherein said first series of equivalent doses is administered according to a three-times-a-week dosing schedule.

17. The method of claim 12, wherein said first series of equivalent doses is administered according to a four-times-a-week dosing schedule.

18. The method of claim 12, wherein said first series of equivalent doses is administered according to a five-times-a-week dosing schedule.

19. The method of claim 12, wherein said first series of equivalent doses is administered according to a five-times-a-week dosing schedule.

20. The method of claim 12, wherein said first series of equivalent doses is administered according to a six-times-a-week dosing schedule.

21. The method of claim 12, wherein said first series of equivalent doses is administered according to a seven-times-a-week dosing schedule.

22. The method of claim 12, wherein said lower total weekly dose of IL-2 is administered as a single dose.

23. The method of claim 12, wherein said second series of equivalent doses is administered according to a two-times-a-week dosing schedule.

24. The method of claim 12, wherein said second series of equivalent doses is administered according to a three-times-a-week dosing schedule.

25. The method of claim 12, wherein said second series of equivalent doses is administered according to a four-times-a-week dosing schedule.

26. The method of claim 12, wherein said second series of equivalent doses is administered according to a five-times-a-week dosing schedule.

27. The method of claim 12, wherein said second series of equivalent doses is administered according to a five-times-a-week dosing schedule.

28. The method of claim 12, wherein said second series of equivalent doses is administered according to a six-times-a-week dosing schedule.

29. The method of claim 12, wherein said second series of equivalent doses is administered according to a seven-times-a-week dosing schedule.

30. The method of claim 1, wherein said higher total weekly dose of IL-2 is in an amount equivalent to a total weekly dose of a reference IL-2 standard in a range from 2000 μg to 3600 μg as determined by the area under the serum concentration-time curve from human pharmacokinetic (PK) data, and wherein said lower total weekly dose of IL-2 is in an amount equivalent to a total weekly dose of a reference IL-2 standard in a range from 1200 μg to about 2600 μg as determined by the area under the serum concentration-time curve from human PK data, and wherein said lower total weekly dose of IL-2 is lower than said higher total weekly dose of IL-2.

31. The method of claim 30, wherein said higher total weekly dose of IL-2 is administered as a single dose or is partitioned into a first series of equivalent doses that are administered according to a two-, three-, four-, five-, six- or seven-times-a-week dosing schedule, and wherein said lower total weekly dose of IL-2 is administered as a single dose or is partitioned into a second series of equivalent doses that are administered according to a two-, three-, four-, five-, six- or seven-times-a-week dosing schedule.

32. The method of claim 30, wherein said higher total weekly dose of IL-2 is 2800 μg and said lower total weekly dose of IL-2 is 2000 μg.

33. The method of claim 30, wherein said therapeutically effective dose of said anti-CD20 antibody is in the range from about 125 mg/m² to about 500 mg/m².

34. The method of claim 1, wherein said higher total weekly dose of IL-2 is in an amount equivalent to a total weekly dose of a reference IL-2 standard in a range from 2000 μg to 3600 μg as determined by the area under the serum concentration-time curve from human PK data, and wherein said lower total weekly dose of IL-2 is in an amount equivalent to a total weekly dose of a reference IL-2 standard in a range from 1200 μg to about 2000 μg as determined by the area under the serum concentration-time curve from human PK data, and wherein said lower total weekly dose of IL-2 is lower than said higher total weekly dose of IL-2.

35. The method of claim 34, wherein said higher total weekly dose of IL-2 is 2800 μg and said lower total weekly dose of IL-2 is 2000 μg.

36. The method of claim 33, wherein said therapeutically effective dose of said anti-CD20 antibody is in the range from about 225 mg/m² to about 400 mg/m².

37. The method of claim 1, wherein said IL-2 is provided in a pharmaceutical composition selected from the group consisting of a monomeric IL-2 pharmaceutical composition, a multimeric IL-2 composition, a stabilized lyophilized IL-2 pharmaceutical composition, and a stabilized spray-dried IL-2 pharmaceutical composition.

38. The method of claim 1, wherein said IL-2 is recombinantly produced IL-2 having an amino acid sequence for human IL-2 or a variant thereof having at least 70% sequence identity to the amino acid sequence for human IL-2.

39. The method of claim 38, wherein said variant there of is des-alanyl-1, serine 125 human interleukin-2.

40. The method of claim 1, wherein said anti-CD20 antibody is an immunologically active anti-CD20 antibody.

41. The method of claim 40, wherein said anti-CD20 antibody is IDEC-C2B8 or fragment thereof.

42. The method of claim 1, wherein said anti-CD20 antibody is a human anti-CD20 antibody, a humanized anti-CD20 antibody, or a chimeric anti-CD20 antibody.

43. The method of claim 1, wherein one or more subsequent maintenance cycles is initiated about 1 month to about 6 months following completion of a first maintenance cycle or completion of any subsequent maintenance cycles.

44. The method of claim 43, wherein natural-killer (NK) cell counts are monitored in said subject to determine when each of said maintenance cycles is initiated, said maintenance cycles being initiated when NK cell count is less than an acceptable threshold level.

45. The method of claim 44, wherein said acceptable threshold level is 200 or less.

46. The method of claim 45, wherein said acceptable threshold level is 150 or less.

47. The method of claim 1, further comprising an interruption in said two-level dosing regimen of IL-2, said interruption comprising a time period off of IL-2 administration between said first time period and said second time period of said two-level dosing regimen of IL-2.

48. The method of claim 47, wherein said interruption further comprises a time period off of anti-CD20 antibody administration.

49. The method of claim 47, wherein natural-killer (NK) cell counts are monitored in said human to determine when said second time period of said two-level dosing regimen is initiated, said second time period being initiated when NK cell count is less than an acceptable threshold level.

50. The method of claim 49, wherein said acceptable threshold level is 200 or less.

51. The method of claim 50, wherein said acceptable threshold level is 150 or less.

52. The method of claim 47, wherein said interruption has a duration of about 1 week to about 4 weeks.

53. A method of treating non-Hodgkin's B-cell lymphoma in a human, said method comprising administering to said human a therapeutically effective dose of anti-CD20 antibody once a week for 4 weeks to 8 weeks beginning on day 1 of a treatment period, and administering a therapeutically effective dose of IL-2 three times a week for 4 weeks to 10 weeks beginning on day 8 of said treatment period, wherein said therapeutically effective dose of anti-CD20 antibody is in the range from about 125 mg/m² to about 500 mg/m², and wherein said therapeutically effective dose of IL-2 is in an amount necessary to achieve the same initial IL-2 exposure as a dose of a reference IL-2 standard in a range from about 666.67 μg to about 1200 μg as determined by the area under the serum concentration-time curve from human PK data.

54. The method of claim 53, wherein said IL-2 is administered subcutaneously.

55. The method of 53, wherein said therapeutically effective dose of said anti-CD20 antibody is administered once a week for 4 weeks, and wherein said therapeutically effective dose of said IL-2 is administered three times a week for 4 weeks or 8 weeks.

56. The method of 53, wherein said therapeutically effective dose of said anti-CD20 antibody is administered once a week for 8 weeks, and wherein said therapeutically effective dose of said IL-2 thereof is administered three times a week for 8 weeks.

57. The method of claim 53, wherein said therapeutically effective dose of said anti-CD20 antibody is in the range from about 225 mg/m² to about 400 mg/m², and wherein said therapeutically effective dose of IL-2 is in an amount necessary to achieve the same initial IL-2 exposure as a dose of a reference IL-2 standard in a range from about 933.33 μg to about 1200 μg as determined by the area under the serum concentration-time curve from human PK data.

58. The method of claim 57, wherein said therapeutically effective dose of said anti-CD20 antibody is about 375 mg/m² and wherein said therapeutically effective dose of IL-2 is in an amount necessary to achieve the same initial IL-2 exposure as a dose of a reference IL-2 standard of about 933.33 μg as determined by the area under the serum concentration-time curve from human PK data.

59. The method of 53, wherein said human is administered a total weekly dose of IL-2 in an amount equivalent to a total weekly dose of a reference IL-2 standard in a range from 2000 μg to 3600 μg as determined by the area under the serum concentration-time curve from human PK data.

60. The method of 59, wherein said total weekly dose of IL-2 is an amount equivalent to a total weekly dose of a reference IL-2 standard in a range from 2800 μg to 3600 μg as determined by the area under the serum concentration-time curve from human PK data.

61. A method for predicting clinical response of a subject undergoing a time period of concurrent therapy with anti-CD20 antibody and IL-2, said method comprising monitoring natural killer (NK) cell expansion in said subject at about 1 week to about 10 weeks post-initiation of said time period of concurrent therapy.

62. The method of claim 61, wherein said time period of concurrent therapy is about 5 weeks, and wherein said monitoring of said NK cell expansion occurs at about 4 weeks to about 10 weeks post-initiation of said time period of concurrent therapy.

63. The method of claim 62, wherein a therapeutically effective dose of said anti-CD20 antibody is administered once per week for a period of 4 weeks starting on day 1 of a treatment period, and a therapeutically effective dose of said IL-2 is administered three times per week for a period of 4 weeks starting on day 8 of said treatment period, and wherein said NK cell expansion is monitored at about 10 weeks post-initiation of said time period of concurrent therapy.

64. The method of 63, wherein said human is administered a total weekly dose of IL-2 in an amount equivalent to a total weekly dose of a reference IL-2 standard in a range from 2000 μg to 3600 μg as determined by the area under the serum concentration-time curve from human PK data.

65. The method of claim 63, wherein said subject has an NK cell count of at least about 170 cells/μl, and wherein said subject is characterized by having a complete response, a partial response, or stable disease.

66. A method for treating non-Hodgkin's B-cell lymphoma in a human subject, comprising administering to said subject at least one therapeutically effective dose of an anti-CD20 antibody and providing a means for maintaining natural-killer (NK) cell count in said subject above an acceptable threshold level, said means comprising administering at least one therapeutically effective dose of interleukin-2 (IL-2) in an amount that results in an initial IL-2 exposure within a range from about 22 IU*hour/ml serum to about 653 IU*hour/ml serum, wherein said IL-2 exposure is measured as the area under the serum concentration-time curve (AUC) as determined by human pharmacokinetic (PK) data.

67. The method of claim 66, wherein said acceptable threshold level is about 150.

68. The method of claim 66, wherein IL-2 is administered according to a constant IL-2 dosing regimen, and wherein said therapeutically effective dose of IL-2 is an amount necessary to achieve the same initial IL-2 exposure as a dose of a reference IL-2 standard in a range from about 666.67 μg to about 1200 μg as determined by the area under the serum concentration-time curve from human PK data.

69. The method of claim 68, wherein said therapeutically effective dose of anti-CD20 antibody is in the range from about 125 mg/m² to about 500 mg/m².

70. The method of claim 68, wherein said constant IL-2 dosing regimen comprises administering said therapeutically effective dose of IL-2 according to a two-times-a-week or three-times-a-week dosing schedule.

71. The method of claim 66, wherein IL-2 is administered according to a two-level dosing regimen of IL-2, wherein said two-level dosing regimen of IL-2 comprises a first time period, wherein a higher total weekly dose of IL-2 is administered to said subject, followed by a second time period, wherein a lower total weekly dose of IL-2 is administered to said subject.

72. The method of claim 71, wherein said higher total weekly dose of IL-2 is in an amount equivalent to a total weekly dose of a reference IL-2 standard in a range from 2000 μg to 3600 μg as determined by the area under the serum concentration-time curve from human pharmacokinetic (PK) data, and wherein said lower total weekly dose of IL-2 is in an amount equivalent to a total weekly dose of a reference IL-2 standard in a range from 1200 μg to about 2600 μg as determined by the area under the serum concentration-time curve from human PK data, and wherein said lower total weekly dose of IL-2 is lower than said higher total weekly dose of IL-2.

73. The method of claim 72, wherein said therapeutically effective dose of anti-CD20 antibody is in the range from about 125 mg/m² to about 500 mg/m².

74. The method of claim 71, wherein a first dose of IL-2 is administered to said subject prior to administering a first dose of anti-CD20 antibody.

75. The method of claim 72, wherein a first dose of IL-2 is administered to said subject concurrently with a first dose of anti-CD20 antibody.

76. The method of claim 72, wherein a first dose of IL-2 is administered to said subject one week after a first dose of anti-CD20 antibody is administered to said subject.

77. The method of claim 72, wherein said higher total weekly dose of IL-2 is administered as a single dose or is partitioned into a first series of equivalent doses that are administered according to a two-, three-, four-, five-, six- or seven-times-a-week dosing schedule, and wherein said lower total weekly dose of IL-2 is administered as a single dose or is partitioned into a second series of equivalent doses that are administered according to a two-, three-, four-, five-, six- or seven-times-a-week dosing schedule.
